(19) United States
(12) Patent Application Publication
(10) Pub. No.: US 2019/0117682 A1
(43) Pub. Date: Apr. 25, 2019

(54) BIO-INSPIRED POLYMERS FOR USE AS SYNTHETIC MUCINS

(71) Applicant: Massachusetts Institute of Technology, Cambridge, MA (US)

(72) Inventors: Wesley G. Chen, Somerville, MA (US); Katharina Ribbeck, Cambridge, MA (US)

(21) Appl. No.: 16/165,968

(22) Filed: Oct. 19, 2018

Related U.S. Application Data
(60) Provisional application No. 62/575,257, filed on Oct. 20, 2017.

(51) Int. Cl. A61K 31/717; C08B 11/20; A01N 43/16; A61P 1/14; A61P 31/00; A23L 33/25; A23L 33/24

(52) U.S. Cl. CPC A61K 31/717; C08B 11/20; A01N 43/16; A23V 2002/00; A61P 31/00; A23L 33/25; A23L 33/24; A61P 1/14

(57) ABSTRACT

Provided are glycosylated polymers that derive from carboxymethyl cellulose and may function as synthetic mucins. The carboxymethyl cellulose backbone of the polymers provide structures amenable to glycosylation by amidation of the carboxyl groups with glycosylamine moieties. The polymers can inhibit microbial virulence and may be useful in a variety of applications, such as treating microbial infections, preventing biofilm formation, and improving gastrointestinal health. Thus, provided are compositions (e.g., pharmaceutical, prebiotic, nutraceutical, food product) comprising the polymers, lubricants comprising the polymers, coatings comprising the polymers, methods of inhibiting microbial virulence, methods of inhibiting and/or preventing biofilm formation, methods of treating and/or preventing infection (e.g., bacterial, protozoan, or fungal infection). Also provided are methods of preparing the polymers described herein.

### RELATED APPLICATION

The present application claims priority under 35 U.S.C. § 119(e) to U.S. provisional application U.S. Ser. No. 62/575,257, filed Oct. 20, 2017, which is incorporated herein by reference.

### BACKGROUND OF THE INVENTION

Mucus is the protective layer that lines all wet epithelial tissue and exhibits remarkable lubricating and anti-virulence properties that protect against infection. The molecules responsible for the unique properties of mucus are mucins, a class of glycoproteins that are heavily glycosylated with complex oligosaccharides. The extended polypeptide structures result in the oligosaccharides protruding from the backbone, resembling a “bottle-brush” appearance. The degree and density of glycosylation, the oligosaccharide composition, and the overall size of the glycoprotein combine to confer a vast spectrum of remarkable physical and physicochemical properties to mucins.

Mucins perform critical functions within biological systems. When coating epithelial surfaces, either as secreted or membrane-anchored forms, mucins maintain a favorable epithelial hydration state, impart important lubrication properties, and act as an anti-microbial barrier to prevent infection at mucosal surfaces, particularly in the oral cavity. The specific mucin structure-activity relationships for each of these functions remain largely unknown, in large part due to lack of access to defined mucin materials of relevant size and composition. Isolation of mucins from natural sources results in mixtures of various glycoforms and degraded products. While these isolated materials provide a useful starting point, they do not allow deciphering of the structure-activity relationship at the molecular level. Understanding these relationships has the potential to lead to rationally designed synthetic mucins for commercial applications ranging from lubricants for food and medicines, antimicrobial coatings, and anticancer vaccines.

Despite advances in polymer chemistry and protein production, there has been limited success in producing materials that mimic the mechanical and functional properties of mucins. An essential property of these polymers is their highly glycosylated backbones. Although specific oligosaccharides have been identified on various biological polymers, it is unclear how individual sugars, the density and the distribution of which directly influence how polymers act as lubricants as well as anti-infectious agents. Therefore, there is a need for polymeric materials that act as synthetic mucins.

### SUMMARY OF THE INVENTION

The present disclosure provides glycosylated polymers (e.g., carboxymethyl cellulose backbone). The polymers mimic the lubricating and/or antivirulence properties of mucins. The technology for producing these polymers is highly modular and allows for the control of polymer size, glycan choice, and coverage density and uniformity. Accordingly, the disclosure provides a tailored design of synthetic mucins for targeted applications in controlling biofilm formation, toxin production, or development of antibiotic resistance in microbes, or as a lubricant/hydrating layer (e.g., on epithelial surfaces).

In one aspect, the present disclosure provides polymers of Formula (I)

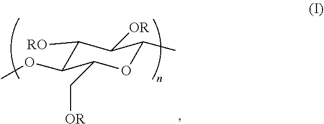

or a salt thereof, wherein:

each occurrence of R is independently hydrogen, —CH₂C(O)OH, —CH₂C(O)OR¹, —CH₂C(O)NHR¹, or R¹;

each occurrence of R¹ is independently a monosaccharide, oligosaccharide, or polysaccharide; and

n is equal to or greater than 20;

wherein the polymer has, on average, about 0.5 to about 2.0 R groups in each repeat unit that are —CH₂C(O)OH, —CH₂C(O)OR¹, or —CH₂C(O)NHR¹.

In certain embodiments, the polymer has a net negative charge at physiological pH.

In certain embodiments, each occurrence of R is independently hydrogen, —CH₂C(O)OH, or —CH₂C(O)NHR¹.

In certain embodiments, R is hydrogen, —CH₂C(O)OH,

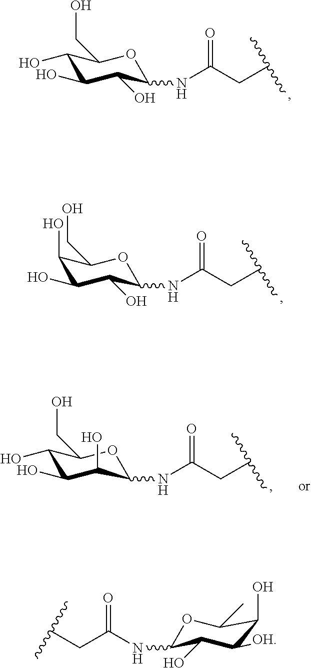

In another aspect, provided is a composition (e.g., pharmaceutical, prebiotic, nutraceutical) comprising a polymer of Formula (I), or a salt thereof, and an acceptable carrier.

In another aspect, provided is a lubricant comprising a polymer of Formula (I), or a salt thereof, or a pharmaceutical composition comprising a polymer of Formula (I).

In another aspect, provided is a method of inhibiting microbial virulence, the method comprising contacting the polymer of Formula (I), or a salt thereof, or a pharmaceutical composition comprising a polymer of Formula (I), with a microbe. In certain embodiments, the microbe is bacteria, archaea, protozoa, fungi or algae.

In another aspect, provided is a method of inhibiting or preventing biofilm formation on a surface, the method comprising coating the surface with the Formula (I), or a salt thereof, or a pharmaceutical composition comprising a polymer of Formula (I). In certain embodiments, the biofilm comprises bacteria, archaea, protozoa, fungi, and/or algae.

In another aspect, provided is a method of treating an infection in a subject in need thereof, the method comprising administering the polymer of Formula (I), or a salt thereof, or a pharmaceutical composition comprising a polymer of Formula (I), to the subject. In certain embodiments, the infection is a bacterial, protozoan, or fungal infection. In certain embodiments, the infection is caused by Streptococcus mutans, Pseudomonas aeruginosa, Staphylococcus aureus, or Candida albicans.

In another aspect, provided is a method of improving the gastrointestinal health of a subject in need thereof, the method comprising administering the polymer of Formula (I), or a salt thereof; a prebiotic composition comprising a polymer of Formula (I), or a salt thereof; or a food product comprising the prebiotic composition, to the subject.

In another aspect, provided is a polymer of Formula (I), or salt thereof, or a pharmaceutical composition comprising the polymer of Formula (I), for use in treating an infection in a subject in need thereof.

In another aspect, provided is a kit comprising a polymer of Formula (I), or a salt thereof, a pharmaceutical composition comprising a polymer of Formula (I), or a salt thereof; a prebiotic composition comprising a polymer of Formula (I), or a salt thereof; or a food product comprising the prebiotic composition. In certain embodiments, the kit further comprises instructions for administration (e.g., human administration) and/or use.

In another aspect, the present disclosure also provides a method of preparing the polymers (e.g., glycosylation of carboxymethyl cellulose).

The details of certain embodiments of the invention are set forth herein. Other features, objects, and advantages of the invention will be apparent from the Detailed Description, Figures, Examples, and Claims.

### Definitions

Definitions of specific functional groups and chemical terms are described in more detail below. The chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Organic Chemistry, Thomas Sorrell, University Science Books, Sausalito, 1999; Smith and March, March's Advanced Organic Chemistry, 5^{th} Edition, John Wiley & Sons, Inc., New York, 2001; Larock, Comprehensive Organic Transformations, VCH Publishers, Inc., New York, 1989; and Carruthers, Some Modern Methods of Organic Synthesis, 3^{rd} Edition, Cambridge University Press, Cambridge, 1987.

Compounds described herein can comprise one or more asymmetric centers, and thus can exist in various stereoisomeric forms, e.g., enantiomers and/or diastereomers. For example, the compounds described herein can be in the form of an individual enantiomer, diastereomer or geometric isomer, or can be in the form of a mixture of stereoisomers, including racemic mixtures and mixtures enriched in one or more stereoisomer. Isomers can be isolated from mixtures by methods known to those skilled in the art, including chiral high pressure liquid chromatography (HPLC) and the formation and crystallization of chiral salts; or preferred isomers can be prepared by asymmetric syntheses. See, for example, Jacques et al., Enantiomers, Racemates and Resolutions (Wiley Interscience, New York, 1981); Wilen et al., Tetrahedron 33:2725 (1977); Eliel, E. L. Stereochemistry of Carbon Compounds (McGraw-Hill, N.Y., 1962); and Wilen, S. H., Tables of Resolving Agents and Optical Resolutions, p. 268 (E. L. Eliel, Ed., Univ. of Notre Dame Press, Notre Dame, Ind. 1972). The invention additionally encompasses compounds as individual isomers substantially free of other isomers, and alternatively, as mixtures of various isomers.

In a formula, [symbol] is a single bond where the stereochemistry of the moieties immediately attached thereto is not specified, - - - is absent or a single bond, and [symbol] or [symbol] is a single or double bond.

Unless otherwise stated, structures depicted herein are also meant to include compounds that differ only in the presence of one or more isotopically enriched atoms. For example, compounds having the present structures except for the replacement of hydrogen by deuterium or tritium, replacement of ¹⁹F with ¹⁸F, or the replacement of ¹²C with ¹³C or ¹⁴C are within the scope of the disclosure. Such compounds are useful, for example, as analytical tools or probes in biological assays.

When a range of values is listed, it is intended to encompass each value and sub-range within the range. For example “C₁₋₆alkyl” is intended to encompass, C₁, C₂, C₃, C₄, C₅, C₆, C₁₋₆, C₁₋₅, C₁₋₄, C₁₋₃, C₁₋₂, C₂₋₆, C₂₋₅, C₂₋₄, C₂₋₃, C₃₋₆, C₃₋₅, C₃₋₄, C₄₋₆, C₄₋₅, and C₅₋₆alkyl.

The term “carbohydrate” or “saccharide” refers to an aldehydic or ketonic derivative of polyhydric alcohols. Carbohydrates include compounds with relatively small molecules (e.g., sugars) as well as macromolecular or polymeric substances (e.g., starch, glycogen, and cellulose polysaccharides). The term “sugar” refers to monosaccharides, oligosaccharides, or polysaccharides. Monosaccharides have a single sugar unit; oligosaccharides have from two to ten sugar units; and polysaccharides have more than ten sugar units. Monosaccharides are the simplest carbohydrates in that they cannot be hydrolyzed to smaller carbohydrates. Most monosaccharides can be represented by the general formula C_{y}H_{2y}O_{y} (e.g., C₆H₁₂O₆ (a hexose such as glucose)), wherein y is an integer equal to or greater than 3. Certain polyhydric alcohols not represented by the general formula described above may also be considered monosaccharides. For example, deoxyribose is of the formula C₅H₁₀O₄ and is a monosaccharide. Monosaccharides usually consist of five or six carbon atoms and are referred to as pentoses and hexoses, receptively. If the monosaccharide contains an aldehyde it is referred to as an aldose; and if it contains a ketone, it is referred to as a ketose. Monosaccharides may also consist of three, four, or seven carbon atoms in an aldose or ketose form and are referred to as trioses, tetroses, and heptoses, respectively. Glyceraldehyde and dihydroxyacetone are considered to be aldotriose and ketotriose sugars, respectively. Examples of aldotetrose sugars include erythrose and threose; and ketotetrose sugars include erythrulose. Aldopentose sugars include ribose, arabinose, xylose, and lyxose; and ketopentose sugars include ribulose, arabulose, xylulose, and lyxulose. Examples of aldohexose sugars include glucose (for example, dextrose), mannose, galactose, allose, altrose, talose, gulose, and idose; and ketohexose sugars include fructose, psicose, sorbose, and tagatose. Ketoheptose sugars include sedoheptulose. Each carbon atom of a monosaccharide bearing a hydroxyl group (OH), with the exception of the first and last carbons, is asymmetric, making the carbon atom a stereocenter with two possible configurations (R or S). Because of this asymmetry, a number of isomers may exist for any given monosaccharide formula. The aldohexose D-glucose, for example, has the formula C₆H₁₂O₆, of which all but two of its six carbons atoms are stereogenic, making D-glucose one of the 16 (i.e., 2⁴) possible stereoisomers. The assignment of D or L is made according to the orientation of the asymmetric carbon furthest from the carbonyl group: in a standard Fischer projection if the hydroxyl group is on the right the molecule is a D sugar, otherwise it is an L sugar. The aldehyde or ketone group of a straight-chain monosaccharide will react reversibly with a hydroxyl group on a different carbon atom to form a hemiacetal or hemiketal, forming a heterocyclic ring with an oxygen bridge between two carbon atoms. Rings with five and six atoms are called furanose and pyranose forms, respectively, and exist in equilibrium with the straight-chain form. During the conversion from the straight-chain form to the cyclic form, the carbon atom containing the carbonyl oxygen, called the anomeric carbon, becomes a stereogenic center with two possible configurations: the oxygen atom may take a position either above or below the plane of the ring. The resulting possible pair of stereoisomers is called anomers. In an α anomer, the —OH substituent on the anomeric carbon rests on the opposite side (trans) of the ring from the —CH₂OH side branch. The alternative form, in which the —CH₂OH substituent and the anomeric hydroxyl are on the same side (cis) of the plane of the ring, is called a β anomer. A carbohydrate including two or more joined monosaccharide units is called an oligosaccharide or polysaccharide, respectively. The two or more monosaccharide units bound together by a covalent bond known as a glycosidic linkage formed via a dehydration reaction, resulting in the loss of a hydrogen atom from one monosaccharide and a hydroxyl group from another. Exemplary disaccharides include sucrose, lactulose, lactose, maltose, isomaltose, trehalose, cellobiose, xylobiose, laminaribiose, gentiobiose, mannobiose, melibiose, nigerose, or rutinose. Exemplary trisaccharides include, but are not limited to, isomaltotriose, nigerotriose, maltotriose, melezitose, maltotriulose, raffinose, and kestose. The term carbohydrate also includes other natural or synthetic stereoisomers of the carbohydrates described herein.

A group is optionally substituted unless expressly provided otherwise. The term “optionally substituted” refers to being substituted or unsubstituted. In certain embodiments, monosaccharide, oligosaccharide, and polysaccharide groups are optionally substituted. “Optionally substituted” refers to a group which may be substituted or unsubstituted (e.g., “substituted” or “unsubstituted” monosaccharide, “substituted” or “unsubstituted” oligosaccharide, “substituted” or “unsubstituted” polysaccharide). In general, the term “substituted” means that at least one hydrogen present on a group is replaced with a permissible substituent, e.g., a substituent which upon substitution results in a stable compound, e.g., a compound which does not spontaneously undergo transformation such as by rearrangement, cyclization, elimination, or other reaction. Unless otherwise indicated, a “substituted” group has a substituent at one or more substitutable positions of the group, and when more than one position in any given structure is substituted, the substituent is either the same or different at each position. The term “substituted” is contemplated to include substitution with all permissible substituents of organic compounds, and includes any of the substituents described herein that results in the formation of a stable compound. The present invention contemplates any and all such combinations in order to arrive at a stable compound. For purposes of this invention, heteroatoms such as nitrogen may have hydrogen substituents and/or any suitable substituent as described herein which satisfy the valencies of the heteroatoms and results in the formation of a stable moiety. The invention is not intended to be limited in any manner by the exemplary substituents described herein.

Exemplary carbon atom substituents include, but are not limited to, halogen, —CN, —NO₂, —N₃, —SO₂H, —SO₃H, —OH, —OR^{aa}, —ON(R^{bb})₂, —N(R^{bb})₃⁺X⁻, —N(OR^{cc})R^{bb}, —SH, —SR^{aa}, —SSR^{cc}, —(═O)R^{aa}, —CO₂H, —CHO, —C(OR^{cc})₂, —CO₂R^{aa}, —OC₂R^{aa}, —OC(═O)R^{aa}, —OCO₂R^{aa}, —C(═O)N(R^{bb})₂, —OC(═O)N(R^{bb})₂, —NR^{bb}C(═O)R^{aa}, —NR^{bb}CO₂R^{aa}, —NR^{bb}C(═O)N(R^{bb})₂, —C(═NR^{bb})R^{aa}, —C(═NR^{bb})OR^{aa}, —OC(═NR^{bb})R^{aa}, —OC(═NR^{bb})OR^{aa}, —C(═NR^{bb})N(R^{bb})₂, —OC(═NR^{bb})N(R^{bb})₂, —NR^{bb}C(═NR^{bb})N(R^{bb})₂, —C(═O)NR^{bb}SO₂R^{aa}, —NR^{bb}SO₂R^{aa}, —SO₂N(R^{bb})₂, —SO₂R^{aa}, —SO₂OR^{aa}, —OSO₂R^{aa}, —S(═O)R^{aa}, —OS(═O)R^{aa}, —Si(R^{aa})₃, —OSi(R^{aa})₃ —C(═S)N(R^{bb})₂, —C(═O)SR^{aa}, —C(═S)SR^{aa}, —SC(═S)SR^{aa}, —SC(═O)SR^{aa}, —OC(═O)SR^{aa}, —SC(═O)OR^{aa}, —SC(═O)R^{aa}, —P(═O)(R^{aa})₂, —P(═O)(OR^{cc})₂, —OP(═O)(R^{aa})₂, —OP(═O)(OR^{cc})₂, —P(═O)(N(R^{bb})₂)₂, —OP(═O)(N(R^{bb})₂)₂, —NR^{bb}P(═O)(R^{aa})₂, —NR^{bb}P(═O)(OR^{cc})₂, —NR^{bb}P(═O)(N(R^{bb})₂)₂), —P(OR^{cc})₂, —P(R^{cc})₃⁺X⁻, —P(OR^{cc})₃⁺X⁻, —P(R^{cc})ᵣ, —P(OR^{cc})₄, —OP(R^{cc})₂, —OP(R^{cc})₃⁺X⁻, —OP(OR^{cc})₂, —OP(OR^{cc})₃⁺X⁻, —OP(R^{cc})₄, —OP(OR^{cc})₄, —B(R^{aa})₂, —B(OR^{cc})₂, —BR^{aa}(OR^{cc}), C₁₋₁₀alkyl, C₁₋₁₀perhaloalkyl, C₂₋₁₀alkenyl, C₂₋₁₀alkynyl, heteroC₁₋₁₀ alkyl, heteroC₂₋₁₀alkenyl, heteroC₂₋₁₀alkynyl, C₃₋₁₀carbocyclyl, 3-14 membered heterocyclyl, C₆₋₁₄aryl, and 5-14 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups; wherein X⁻ is a counterion;

or two geminal hydrogens on a carbon atom are replaced with the group ═O, ═S, ═NN(R^{bb})₂, ═NNR^{bb}C(═O)R^{aa}, ═NNR^{bb}C(═O)OR^{aa}, ═NNR^{bb}S (═O)₂R^{aa}, ═NR^{bb} , or ═NOR^{cc};

each instance of R^{aa} is, independently, selected from C₁₋₁₀ alkyl, C₁₋₁₀ perhaloalkyl, C₂₋₁₀ alkenyl, C₂₋₁₀alkynyl, heteroC₁₋₁₀alkyl, heteroC₂₋₁₀alkenyl, heteroC₂₋₁₀alkynyl, C₃₋₁₀carbocyclyl, 3-14 membered heterocyclyl, C₆₋₁₄aryl, and 5-14 membered heteroaryl, or two R^{aa} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups;

each instance of R^{bb} is, independently, selected from hydrogen, —OH, —OR^{aa}, —N(R^{cc})₂, —CN, —C(═O)R^{aa}, —C(═O)N(R^{cc})₂, —CO₂R^{aa}, —SO₂R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, —P(═O)(R^{aa})₂, —P(═O)(OR^{cc})₂, —P(═O)(N(R^{cc})₂)₂, C₁₋₁₀alkyl, C₁₋₁₀perhaloalkyl, C₂₋₁₀alkenyl, C₁₋₁₀alkynyl, heteroC₁₋₁₀alkyl, heteroC₂₋₁₀alkenyl, heteroC₂₋₁₀alkynyl, C₃₋₁₀carbocyclyl, 3-14 membered heterocyclyl, C₆₋₁₄aryl, and 5-14 membered heteroaryl, or two R^{bb} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups; wherein X⁻ is a counterion;

each instance of RCC is, independently, selected from hydrogen, C₁₋₁₀alkyl, C₁₋₁₀ perhaloalkyl, C₂₋₁₀alkenyl, C₂₋₁₀ alkynyl, heteroC₁₋₁₀alkyl, heteroC₂₋₁₀alkenyl, heteroC₂₋₁₀ alkynyl, C₃₋₁₀carbocyclyl, 3-14 membered heterocyclyl, C₆₋₁₄aryl, and 5-14 membered heteroaryl, or two RCC groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups;

each instance of R^{dd} is, independently, selected from halogen, —CN, —NO₂, —N₃, —SO₂H, —SO₃H, —OH, —OR^{ee}, —ON(R^{ff})₂, —N(R^{ff})₂, —N(R^{ff})₃⁺X³¹ , —N(OR^{ee})R^{ff}, —SH, —SR^{ee}, —SSR^{ee}, —C(═O)R^{ee}, —CO₂H, —CO₂R^{ee}, —OC(═O)R^{ee}, —OCO₂R^{ee}, —C(═O)N(R^{ff})₂, —OC(═O)N(R^{ff})₂, —NR^{ff}C(═O)R^{ee}, —NR^{ff}CO₂R^{ee}, —NR^{ff}C(═O)N(R^{ff})₂, —C(═NR^{ff})OR^{ee}, —OC(═NR^{ff})R^{ee}, —OC(═NR^{ff})OR^{ee}, —C(═NR^{ff})N(R^{ff})₂, —OC(═NR^{ff})N(R^{ff})₂, —NR^{ff}C(═NR^{ff})N(R^{ff})₂, —NR^{ff}SO₂R^{ee}, —SO₂N(R^{ff})₂, —SO₂R^{ee}, —SO₂OR^{ee}, —OSO₂R^{ee}, —S(═O)R^{ee}, —Si(R^{ee})₃, —OSi(R^{ee})₃, —C(═S)N(R^{ff})₂, —C(═O)SR^{ee}, —C(═S)SR^{ee}, —SC(═S)SR^{ee}, —P(═O)(OR^{ee})₂, —P(═O)(R^{ee})₂, —OP(═O)(R^{ee})₂, —OP(═O)(OR^{ee})₂, C₁₋₆alkyl, C₁₋₆perhaloalkyl, C₂₋₆alkenyl, C₂₋₆alkynyl, heteroC₁₋₆alkyl, heteroC₂₋₆alkenyl, heteroC₂₋₆alkynyl, C₃₋₁₀carbocyclyl, 3-10 membered heterocyclyl, C₆₋₁₀aryl, 5-10 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups, or two geminal R^{dd} substituents can be joined to form ═O or ═S; wherein X⁻ is a counterion;

each instance of R^{ee} is, independently, selected from C₁₋₆alkyl, C₁₋₆perhaloalkyl, C₂₋₆ alkenyl, C₂₋₆alkynyl, heteroC₁₋₆alkyl, heteroC₂₋₆alkenyl, heteroC₂₋₆alkynyl, C₃₋₁₀carbocyclyl, C₆₋₁₀aryl, 3-10 membered heterocyclyl, and 3-10 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups;

each instance of R^{ff} is, independently, selected from hydrogen, C₁₋₆alkyl, C₁₋₆ perhaloalkyl, C₂₋₆alkenyl, C₂₋₆alkynyl, heteroC₁₋₆alkyl, heteroC₂₋₆alkenyl, heteroC₂₋₆alkynyl, C₃₋₁₀carbocyclyl, 3-10 membered heterocyclyl, C₆₋₁₀aryl and 5-10 membered heteroaryl, or two Rff groups are joined to form a 3-10 membered heterocyclyl or 5-10 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups; and

each instance of R^{gg} is, independently, halogen, —CN, —NO₂, —N₃, —SO₂H, —SO₃H, —OH, —OC₁₋₆alkyl, —ON(C₁₋₆alkyl)₂, —N(C₁₋₆alkyl)₂, —N(C₁₋₆alkyl)₃⁺X⁻, —NH(C₁₋₆alkyl)₂⁺X⁻, —NH₂(C₁₋₆alkyl)⁺X⁻, —NH₃⁺X⁻, —N(OC₁₋₆alkyl)(C₁₋₆alkyl), —N(OH)(C₁₋₆alkyl), —NH(OH), —SH, —SC₁₋₆alkyl, —SS(C₁₋₆alkyl), —C(═O)(C₁₋₆alkyl), —CO₂H, —CO₂(C₁₋₆alkyl), —OC(═O)(C₁₋₆alkyl), —OCO₂(C₁₋₆alkyl), —C(═O)NH₂, —C(═O)N(C₁₋₆alkyl)₂, —OC(═O)NH(C₁₋₆alkyl), —NHC(═O)(C₁₋₆alkyl), —N(C₁₋₆alkyl)C(═O)(C₁₋₆alkyl), —NHCO₂(C₁₋₆alkyl), —NHC(═O)N(C₁₋₆alkyl)₂, —NHC(═O)NH(C₁₋₆alkyl), —NHC(═O)NH₂, —C(═NH)O(C₁₋₆alkyl), —OC(═NH)(C₁₋₆alkyl), —OC(═NH)OC₁₋₆alkyl, —C(═NH)N(C₁₋₆alkyl)₂, —C(═NH)NH(C₁₋₆alkyl), —C(═NH)NH₂, —OC(═NH)N(C₁₋₆alkyl)₂, —OC(NH)NH(C₁₋₆alkyl), —OC(NH)NH₂, —NHC(NH)N(C₁₋₆alkyl)₂, —NHC(═NH)NH₂, —NHSO₂(C₁₋₆alkyl), —SO₂N(C₁₋₆alkyl)₂, —SO₂NH(C₁₋₆alkyl), —SO₂NH₂, —SO₂C₁₋₆alkyl, —SO₂OC₁₋₆alkyl, —OSO₂C₁₋₆alkyl, —SOC₁₋₆ alkyl, —Si(C₁₋₆alkyl)₃, —OSi(C₁₋₆alkyl)₃ —C(═S)N(C₁₋₆alkyl)₂, C(═S)NH(C₁₋₆alkyl), C(═S)NH₂, —C(═O)S(C₁₋₆alkyl), —C(═S)SC₁₋₆alkyl, —SC(═S)SC₁₋₆alkyl, —P(═O)(OC₁₋₆alkyl)₂, —P(═O)(C₁₋₆alkyl)₂, —OP(═O)(C₁₋₆alkyl)₂, —OP(═O)(OC₁₋₆alkyl)₂, C₁₋₆alkyl, C₁₋₆perhaloalkyl, C₂₋₆alkenyl, C₂₋₆alkynyl, heteroC₁₋₆alkyl, heteroC₂₋₆alkenyl, heteroC₂₋₆alkynyl, C₃₋₁₀carbocyclyl, C₆₋₁₀aryl, 3-10 membered heterocyclyl, 5-10 membered heteroaryl; or two geminal R^{gg} substituents can be joined to form ═O or ═S; wherein X⁻ is a counterion.

The term “amino” refers to the group —NH₂. The term “substituted amino,” by extension, refers to a monosubstituted amino, a disubstituted amino, or a trisubstituted amino. In certain embodiments, the “substituted amino” is a monosubstituted amino or a disubstituted amino group.

Nitrogen atoms can be substituted or unsubstituted as valency permits, and include primary, secondary, tertiary, and quaternary nitrogen atoms. Exemplary nitrogen atom substituents include, but are not limited to, hydrogen, —OH, —OR^{aa}, —N(R^{cc})₂, —CN, —C(═O)R^{aa}, —C(═O)N(R^{cc})₂, —CO₂R^{aa}, —SO₂R^{aa}, —C(═NR^{bb})R^{aa}, —C(═NR^{cc})OR^{aa}, —C(═NR^{cc})N(R^{c})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, —P(═O)(OR^{cc})₂, —P(═O)(R^{aa})₂, —P(═O)(N(R^{cc})₂)₂, C₁₋₁₀alkyl, C₁₋₁₀perhaloalkyl, C₂₋₁₀alkenyl, C₂₋₁₀alkynyl, heteroC₁₋₁₀alkyl, heteroC₂₋₁₀alkenyl, heteroC₂₋₁₀alkynyl, C₃₋₁₀carbocyclyl, 3-14 membered heterocyclyl, C₆₋₁₄aryl, and 5-14 membered heteroaryl, or two R^{cc} groups attached to an N atom are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups, and wherein R^{aa}, R^{bb}, R^{cc} and R^{dd} are as defined above.

In certain embodiments, the substituent present on the nitrogen atom is a nitrogen protecting group (also referred to herein as an “amino protecting group”). Nitrogen protecting groups include, but are not limited to, —OH, —OR^{aa}, —N(R^{aa})₂, —C(═O)Raa, —C(═O)N(R^{cc})₂, —CO₂R^{aa}, —SO₂R^{aa}, —C(═NR^{cc})R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, C₁₋₁₀alkyl (e.g., aralkyl, heteroaralkyl), C₂₋₁₀alkenyl, C₂₋₁₀alkynyl, heteroC₁₀alkyl, heteroC₂₋₁₀alkenyl, heteroC₂₋₁₀alkynyl, C₃₋₁₀carbocyclyl, 3-14 membered heterocyclyl, C₆₋₁₄aryl, and 5-14 membered heteroaryl groups, wherein each alkyl, alkenyl, alkynyl, heteroalkyl, heteroalkenyl, heteroalkynyl, carbocyclyl, heterocyclyl, aralkyl, aryl, and heteroaryl is independently substituted with 0, 1, 2, 3, 4, or 5 R^{dd} groups, and wherein R^{aa}, R^{bb}, R^{cc} and R^{dd} are as defined herein. Nitrogen protecting groups are well known in the art and include those described in detail in Protecting Groups in Organic Synthesis, T. W. Greene and P. G. M. Wuts, 3^{rd} edition, John Wiley & Sons, 1999, incorporated herein by reference.

For example, nitrogen protecting groups such as amide groups (e.g., —C(═O)R^{aa}) include, but are not limited to, formamide, acetamide, chloroacetamide, trichloroacetamide trifluoroacetamide, phenylacetamide, 3-phenylpropanamide, picolinamide, 3-pyridylcarboxamide, N-benzoylphenylalanyl derivative, benzamide, p-phenylbenzamide, o-nitophenylacetamide, o-nitrophenoxyacetamide, acetoacetamide, (N′-dithiobenzyloxyacylamino)acetamide, 3-(p-hydroxyphenyl)propanamide, 3-(o-nitrophenyl)propanamide, 2-methyl-2-(o-nitrophenoxy)propanamide, 2-methyl-2-(o-phenylazophenoxy)propanamide, 4-chlorobutanamide, 3-methyl-3-nitrobutanamide, o-nitrocinnamide, N-acetylmethionine derivative, o-nitrobenzamide and o-(benzoyloxymethyl)benzamide.

Nitrogen protecting groups such as carbamate groups (e.g., —C(═O)OR^{aa}) include, but are not limited to, methyl carbamate, ethyl carbamate, 9-fluorenylmethyl carbamate (Fmoc), 9-(2-sulfo)fluorenylmethyl carbamate, 9-(2,7-dibromo)fluoroenylmethyl carbamate, 2,7-di-t-butyl-[9-(10,10-dioxo-10,10,10,10-tetrahydrothioxanthyl]methyl carbamate (DBD-Tmoc), 4-methoxyphenacyl carbamate (Phenoc), 2,2,2-trichloroethyl carbamate (Troc), 2-trimethylsilylethyl carbamate (Teoc), 2-phenylethyl carbamate (hZ), 1-(1-adamantyl)-1-methylethyl carbamate (Adpoc), 1,1-dimethyl-2-haloethyl carbamate, 1,1-dimethyl-2,2-dibromoethyl carbamate (DB-t-BOC), 1,1-dimethyl-2,2,2-trichloroethyl carbamate (TCBOC), 1-methyl-1-(4-biphenylyl)ethyl carbamate (Bpoc), 1-(3,5-di-t-butylphenyl)-1-methylethyl carbamate (t-Bumeoc), 2-(2′- and 4′-pyridyl)ethyl carbamate (Pyoc), 2-(N,N-dicyclohexylcarboxamido)ethyl carbamate, t-butyl carbamate (BOC or Boc), 1-adamantyl carbamate (Adoc), vinyl carbamate (Voc), allyl carbamate (Alloc), 1-isopropylallyl carbamate (Ipaoc), cinnamyl carbamate (Coc), 4-nitrocinnamyl carbamate (Noc), 8-quinolyl carbamate, N-hydroxypiperidinyl carbamate, alkyldithio carbamate, benzyl carbamate (Cbz), p-methoxybenzyl carbamate (Moz), p-nitobenzyl carbamate, p-bromobenzyl carbamate, p-chlorobenzyl carbamate, 2,4-dichlorobenzyl carbamate, 4-methylsulfinylbenzyl carbamate (Msz), 9-anthrylmethyl carbamate, diphenylmethyl carbamate, 2-methylthioethyl carbamate, 2-methylsulfonylethyl carbamate, 2-(p-toluenesulfonyl)ethyl carbamate, [2-(1,3-dithianyl)]methyl carbamate (Dmoc), 4-methylthiophenyl carbamate (Mtpc), 2,4-dimethylthiophenyl carbamate (Bmpc), 2-phosphonioethyl carbamate (Peoc), 2-triphenylphosphonioisopropyl carbamate (Ppoc), 1,1-dimethyl-2-cyanoethyl carbamate, m-chloro-p-acyloxybenzyl carbamate, p-(dihydroxyboryl)benzyl carbamate, 5-benzisoxazolylmethyl carbamate, 2-(trifluoromethyl)-6-chromonylmethyl carbamate (Tcroc), m-nitrophenyl carbamate, 3,5-dimethoxybenzyl carbamate, o-nitrobenzyl carbamate, 3,4-dimethoxy-6-nitrobenzyl carbamate, phenyl(o-nitrophenyl)methyl carbamate, t-amyl carbamate, S-benzyl thiocarbamate, p-cyanobenzyl carbamate, cyclobutyl carbamate, cyclohexyl carbamate, cyclopentyl carbamate, cyclopropylmethyl carbamate, p-decyloxybenzyl carbamate, 2,2-dimethoxyacylvinyl carbamate, o-(N,N-dimethylcarboxamido)benzyl carbamate, 1,1-dimethyl-3-(N,N-dimethylcarboxamido)propyl carbamate, 1,1-dimethylpropynyl carbamate, di(2-pyridyl)methyl carbamate, 2-furanylmethyl carbamate, 2-iodoethyl carbamate, isoborynl carbamate, isobutyl carbamate, isonicotinyl carbamate, p-(p′-methoxyphenylazo)benzyl carbamate, 1-methylcyclobutyl carbamate, 1-methylcyclohexyl carbamate, 1-methyl-1-cyclopropylmethyl carbamate, 1-methyl-1-(3,5-dimethoxyphenyl)ethyl carbamate, 1-methyl-1-(p-phenylazophenyl)ethyl carbamate, 1-methyl-1-phenylethyl carbamate, 1-methyl-1-(4-pyridyl)ethyl carbamate, phenyl carbamate, p-(phenylazo)benzyl carbamate, 2,4,6-tri-t-butylphenyl carbamate, 4-(trimethylammonium)benzyl carbamate, and 2,4,6-trimethylbenzyl carbamate.

Nitrogen protecting groups such as sulfonamide groups (e.g., S(═O)₂R^{aa}) include, but are not limited to, p-toluenesulfonamide (Ts), benzenesulfonamide, 2,3,6-trimethyl-4-methoxybenzenesulfonamide (Mtr), 2,4,6-trimethoxybenzenesulfonamide (Mtb), 2,6-dimethyl-4-methoxybenzenesulfonamide (Pme), 2,3,5,6-tetramethyl-4-methoxybenzenesulfonamide (Mte), 4-methoxybenzenesulfonamide (Mbs), 2,4,6-trimethylbenzenesulfonamide (Mts), 2,6-dimethoxy-4-methylbenzenesulfonamide (iMds), 2,2,5,7,8-pentamethylchroman-6-sulfonamide (Pmc), methanesulfonamide (Ms), β-trimethylsilylethanesulfonamide (SES), 9-anthracenesulfonamide, 4-(4′,8′-dimethoxynaphthylmethyl)benzenesulfonamide (DNMBS), benzylsulfonamide, trifluoromethylsulfonamide, and phenacylsulfonamide. Other nitrogen protecting groups include, but are not limited to, phenothiazinyl-(10)-acyl derivative, N′-p-toluenesulfonylaminoacyl derivative, N′-phenylaminothioacyl derivative, N-benzoylphenylalanyl derivative, N-acetylmethionine derivative, 4,5-diphenyl-3-oxazolin-2-one, N-phthalimide, N-dithiasuccinimide (Dts), N-2,3-diphenylmaleimide, N-2,5-dimethylpyrrole, N-1,1,4,4-tetramethyldisilylazacyclopentane adduct (STABASE), 5-substituted 1,3-dimethyl-1,3,5-triazacyclohexan-2-one, 5-substituted 1,3-dibenzyl-1,3,5-triazacyclohexan-2-one, 1-substituted 3,5-dinitro-4-pyridone, N-methylamine, N-allylamine, N-[2-(trimethylsilyl)ethoxy]methylamine (SEM), N-3-acetoxypropylamine, N-(1-isopropyl-4-nitro-2-oxo-3-pyroolin-3-yl)amine, quaternary ammonium salts, N-benzylamine, N-di(4-methoxyphenyl)methylamine, N-5-dibenzosuberylamine, N-triphenylmethylamine (Tr), N-[(4-methoxyphenyl)diphenylmethyl]amine (MMTr), N-9-phenylfluorenylamine (PhF), N-2,7-dichloro-9-fluorenylmethyleneamine, N-ferrocenylmethylamino (Fcm), N-2-picolylamino N′-oxide, N-1,1-dimethylthiomethyleneamine, N-benzylideneamine, N-p-methoxybenzylideneamine, N-diphenylmethyleneamine, N-[(2-pyridyl)mesityl]methyleneamine, N-(N′,N′-dimethylaminomethylene)amine, N,N′-isopropylidenediamine, N-p-nitrobenzylideneamine, N-salicylideneamine, N-5-chlorosalicylideneamine, N-(5-chloro-2-hydroxyphenyl)phenylmethyleneamine, N-cyclohexylideneamine, N-(5,5-dimethyl-3-oxo-1-cyclohexenyl)amine, N-borane derivative, N-diphenylborinic acid derivative, N-[phenyl(pentaacylchromium- or tungsten)acyl]amine, N-copper chelate, N-zinc chelate, N-nitroamine, N-nitrosoamine, amine N-oxide, diphenylphosphinamide (Dpp), dimethylthiophosphinamide (Mpt), diphenylthiophosphinamide (Ppt), dialkyl phosphoramidates, dibenzyl phosphoramidate, diphenyl phosphoramidate, benzenesulfenamide, o-nitrobenzenesulfenamide (Nps), 2,4-dinitrobenzenesulfenamide, pentachlorobenzenesulfenamide, 2-nitro-4-methoxybenzenesulfenamide, triphenylmethylsulfenamide, and 3-nitropyridinesulfenamide (Npys).

In certain embodiments, the substituent present on an oxygen atom is an oxygen protecting group (also referred to herein as an “hydroxyl protecting group”). Oxygen protecting groups include, but are not limited to, —R^{aa}, —N(R^{bb})₂, —C(═O)SR^{aa}, —C(═O)R^{aa}, —CO₂R^{aa}, —C(═O)N(R^{bb})₂, —C(═NR^{bb})R^{aa}, —C(═NR^{bb})OR^{aa}, 13 C(═NR^{bb})N(R^{bb})₂, —S(═O)R^{aa}, —SO₂R^{aa}, —Si(R^{aa})₃, —P(R^{cc})₂, —P(R^{cc})₃⁺X⁻, —P(OR^{cc})₂, —P(OR^{cc})₃⁺X⁻, —P(═O)(R^{aa})₂, —P(═O)(OR^{c})₂, and —P(═O)(N(R^{bb})₂)₂, wherein X⁻, R^{aa}, R^{bb}, and R^{cc} are as defined herein. Oxygen protecting groups are well known in the art and include those described in detail in Protecting Groups in Organic Synthesis, T. W. Greene and P. G. M. Wuts, 3^{rd} edition, John Wiley & Sons, 1999, incorporated herein by reference.

Exemplary oxygen protecting groups include, but are not limited to, methyl, methoxylmethyl (MOM), methylthiomethyl (MTM), t-butylthiomethyl, (phenyldimethylsilyl)methoxymethyl (SMOM), benzyloxymethyl (BOM), p-methoxybenzyloxymethyl (PMBM), (4-methoxyphenoxy)methyl (p-AOM), guaiacolmethyl (GUM), t-butoxymethyl, 4-pentenyloxymethyl (POM), siloxymethyl, 2-methoxyethoxymethyl (MEM), 2,2,2-trichloroethoxymethyl, bis(2-chloroethoxy)methyl, 2-(trimethylsilyl)ethoxymethyl (SEMOR), tetrahydropyranyl (THP), 3-bromotetrahydropyranyl, tetrahydrothiopyranyl, 1-methoxycyclohexyl, 4-methoxytetrahydropyranyl (MTHP), 4-methoxytetrahydrothiopyranyl, 4-methoxytetrahydrothiopyranyl S,S-dioxide, 1-[(2-chloro-4-methyl)phenyl]-4-methoxypiperidin-4-yl (CTMP), 1,4-dioxan-2-yl, tetrahydrofuranyl, tetrahydrothiofuranyl, 2,3,3a,4,5,6,7,7a-octahydro-7,8,8-trimethyl-4,7-methanobenzofuran-2-yl, 1-ethoxyethyl, 1-(2-chloroethoxy)ethyl, 1-methyl-1-methoxyethyl, 1-methyl-1-benzyloxyethyl, 1-methyl-1-benzyloxy-2-fluoroethyl, 2,2,2-trichloroethyl, 2-trimethylsilylethyl, 2-(phenylselenyl)ethyl, t-butyl, allyl, p-chlorophenyl, p-methoxyphenyl, 2,4-dinitrophenyl, benzyl (Bn), p-methoxybenzyl, 3,4-dimethoxybenzyl, o-nitrobenzyl, p-nitrobenzyl, p-halobenzyl, 2,6-dichlorobenzyl, p-cyanobenzyl, p-phenylbenzyl, 2-picolyl, 4-picolyl, 3-methyl-2-picolyl N-oxido, diphenylmethyl, p,p′-dinitrobenzhydryl, 5-dibenzosuberyl, triphenylmethyl, α-naphthyldiphenylmethyl, p-methoxyphenyldiphenylmethyl, di(p-methoxyphenyl)phenylmethyl, tri(p-methoxyphenyl)methyl, 4-(4′-bromophenacyloxyphenyl)diphenylmethyl, 4,4′,4″-tris(4,5-dichlorophthalimidophenyl)methyl, 4,4′,4″-tris(levulinoyloxyphenyl)methyl, 4,4′,4″-tris(benzoyloxyphenyl)methyl, 3-(imidazol-1-yl)bis(4′,4″-dimethoxyphenyl)methyl, 1,1-bis(4-methoxyphenyl)-1′-pyrenylmethyl, 9-anthryl, 9-(9-phenyl)xanthenyl, 9-(9-phenyl-10-oxo)anthryl, 1,3-benzodithiolan-2-yl, benzisothiazolyl S,S-dioxido, trimethylsilyl (TMS), triethylsilyl (TES), triisopropylsilyl (TIPS), dimethylisopropylsilyl (IPDMS), diethylisopropylsilyl (DEIPS), dimethylthexylsilyl, t-butyldimethylsilyl (TBDMS), t-butyldiphenylsilyl (TBDPS), tribenzylsilyl, tri-p-xylylsilyl, triphenylsilyl, diphenylmethylsilyl (DPMS), t-butylmethoxyphenylsilyl (TBMPS), formate, benzoylformate, acetate, chloroacetate, dichloroacetate, trichloroacetate, trifluoroacetate, methoxyacetate, triphenylmethoxyacetate, phenoxyacetate, p-chlorophenoxyacetate, 3-phenylpropionate, 4-oxopentanoate (levulinate), 4,4-(ethylenedithio)pentanoate (levulinoyldithioacetal), pivaloate, adamantoate, crotonate, 4-methoxycrotonate, benzoate, p-phenylbenzoate, 2,4,6-trimethylbenzoate (mesitoate), methyl carbonate, 9-fluorenylmethyl carbonate (Fmoc), ethyl carbonate, 2,2,2-trichloroethyl carbonate (Troc), 2-(trimethylsilyl)ethyl carbonate (TMSEC), 2-(phenylsulfonyl) ethyl carbonate (Psec), 2-(triphenylphosphonio) ethyl carbonate (Peoc), isobutyl carbonate, vinyl carbonate, allyl carbonate, t-butyl carbonate (BOC or Boc), p-nitrophenyl carbonate, benzyl carbonate, p-methoxybenzyl carbonate, 3,4-dimethoxybenzyl carbonate, o-nitrobenzyl carbonate, p-nitrobenzyl carbonate, S-benzyl thiocarbonate, 4-ethoxy-1-napththyl carbonate, methyl dithiocarbonate, 2-iodobenzoate, 4-azidobutyrate, 4-nitro-4-methylpentanoate, o-(dibromomethyl)benzoate, 2-formylbenzenesulfonate, 2-(methylthiomethoxy)ethyl, 4-(methylthiomethoxy)butyrate, 2-(methylthiomethoxymethyl)benzoate, 2,6-dichloro-4-methylphenoxyacetate, 2,6-dichloro-4-(1,1,3,3-tetramethylbutyl)phenoxyacetate, 2,4-bis(1,1-dimethylpropyl)phenoxyacetate, chlorodiphenylacetate, isobutyrate, monosuccinoate, (E)-2-methyl-2-butenoate, o-(methoxyacyl)benzoate, α-naphthoate, nitrate, alkyl N,N,N′,N′-tetramethylphosphorodiamidate, alkyl N-phenylcarbamate, borate, dimethylphosphinothioyl, alkyl 2,4-dinitrophenylsulfenate, sulfate, methanesulfonate (mesylate), benzylsulfonate, and tosylate (Ts).

In certain embodiments, the substituent present on a sulfur atom is a sulfur protecting group (also referred to as a “thiol protecting group”). Sulfur protecting groups include, but are not limited to, R^{aa}, N(R^{bb})₂, C(═O)SR^{aa}, C(═O)R^{aa}, CO₂R^{aa}, C(═O)N(R^{bb})₂, —C(═NR^{bb})R^{aa}, —C(═NR^{bb})OR^{aa}, —C(═NR^{bb})N(R^{bb})₂, S(═O)R^{aa}, —SO₂R^{aa}, —Si(R^{aa})₃, —P(R^{cc})₂, —P(R^{cc})₃⁺X⁻, —P(OR^{cc})₂, —P(OR^{cc})₃⁺X⁻, —P(═O)(R^{aa})₂, —P(═O)(OR^{cc})₂, and —P(═O)(N(R^{bb})2)₂, wherein R^{aa}, R^{bb}, and R^{cc} are as defined herein. Sulfur protecting groups are well known in the art and include those described in detail in Protecting Groups in Organic Synthesis, T. W. Greene and P. G. M. Wuts, 3^{rd} edition, John Wiley & Sons, 1999, incorporated herein by reference.

A “counterion” or “anionic counterion” is a negatively charged group associated with a positively charged group in order to maintain electronic neutrality. An anionic counterion may be monovalent (i.e., including one formal negative charge). An anionic counterion may also be multivalent (i.e., including more than one formal negative charge), such as divalent or trivalent. Exemplary counterions include halide ions (e.g., F⁻, Cl⁻, Br, I⁻), NO₃, ClO₄, OH, H₂P₄⁻, HCO₃⁻, HSOᵣ⁻, sulfonate ions (e.g., methansulfonate, trifluoromethanesulfonate, p-toluenesulfonate, benzenesulfonate, 10-camphor sulfonate, naphthalene-2-sulfonate, naphthalene-1-sulfonic acid-5-sulfonate, ethan-1-sulfonic acid-2-sulfonate, and the like), carboxylate ions (e.g., acetate, propanoate, benzoate, glycerate, lactate, tartrate, glycolate, gluconate, and the like), BF₄⁻, PF₄⁻, PF₆⁻, AsF₆⁻, SbF₆⁻, B[3,5-(CF₃)₂C₆H₃]₄]⁻, B(C₆F₅)₄⁻, BPh₄⁻, Al(OC(CF₃)₃)₄⁻, and carborane anions (e.g., CB₁₁H₁₂⁻ or (HCB₁₁Me₅Br₆)⁻). Exemplary counterions which may be multivalent include CO₃²⁻, HPO₄²⁻, PO₄³⁻, B₄O₇²⁻, SO₄²⁻, S₂O₃²⁻, carboxylate anions (e.g., tartrate, citrate, fumarate, maleate, malate, malonate, gluconate, succinate, glutarate, adipate, pimelate, suberate, azelate, sebacate, salicylate, phthalates, aspartate, glutamate, and the like), and carboranes.

The term “polymer” refers to a molecule including two or more (e.g., 3 or more, 4 or more, 5 or more, 10 or more) repeating units which are covalently bound together. In certain embodiments, a polymer comprises 3 or more, 5 or more, 10 or more, 50 or more, 100 or more, 1000 or more, 2000 or more, or 4000 or more repeating units. In certain embodiments, a polymer comprises more than 4000 repeating units. The repeating units of a polymer are referred to as “monomers.” A “homopolymer” is a polymer that consists of a single repeating monomer. A “copolymer” is a polymer that comprises two or more different monomer subunits. Copolymers include, but are not limited to, random, block, alternating, segmented, linear, branched, grafted, and tapered copolymers. Polymers may be natural (e.g., naturally occurring polypeptides), or synthetic (e.g., non-naturally occurring). A polymer may have an overall molecular weight of 50 Da or greater, 100 Da or greater, 500 Da or greater, 1000 Da or greater, 2000 Da or greater, 5000 Da or greater, 10000 Da or greater, 20000 Da or greater, or 50000 Da or greater.

The terms “number average molecular weight,” “number average molar mass,” and “Mₑ” are measurements of the molecular mass of a polymer. The number average molecular mass is the ordinary arithmetic mean or average of the molecular masses of the individual polymers. It is determined by measuring the molecular mass of n polymer molecules, summing the masses, and dividing by n. For example, a polymer having 100 repeating units of a monomer with a molecular weight of 100 g/mol would have a number average molecular weight (Mₙ) of 10,000 g/mol [Mn=(100)*(100 g/mol)/ (1)=10,000 g/mol)]. The number average molar mass of a polymer can be determined by gel permeation chromatography, viscometry via the Mark Houwink equation, colligative methods such as vapor pressure osmometry, end-group determination, or ¹H NMR.

The term “biofilm” refers to any group of microorganisms in which cells attach to each other and often also to a surface. These adherent cells are frequently embedded within a self-produced matrix of extracellular polymeric substance (EPS) which may also be referred to as slime (although not everything described as slime is a biofilm). Biofilm is a polymeric conglomeration generally composed of extracellular DNA, proteins, and polysaccharides. Biofilms may form on living or non-living surfaces and can be prevalent in natural, industrial and hospital settings. The microbial cells growing in a biofilm are physiologically distinct from planktonic cells of the same organism, which, by contrast, are single-cells that may float or swim in a liquid medium. Biofilms can be present on the teeth of most animals as dental plaque, where they may cause tooth decay and gum disease. Microbes form a biofilm in response to many factors, which may include cellular recognition of specific or non-specific attachment sites on a surface, nutritional cues, or in some cases, by exposure of planktonic cells to sub-inhibitory concentrations of antibiotics. When a cell switches to the biofilm mode of growth, it undergoes a phenotypic shift in behavior in which large suites of genes are differentially regulated.

Biofilms are usually found on solid substrates submerged in or exposed to an aqueous solution, although they can form as floating mats on liquid surfaces and also on the surface of leaves, particularly in high humidity climates. Given sufficient resources for growth, a biofilm will quickly grow to be macroscopic (visible to the naked eye). Biofilms can contain many different types of microorganism, e.g., bacteria, archaea, protozoa, fungi, and/or algae; each group performs specialized metabolic functions. However, some organisms will form single-species films under certain conditions. The social structure (cooperation/competition) within a biofilm depends highly on the different species present.

The term “natural transformation” refers to a bacterial adaptation for DNA transfer that depends on the expression of numerous bacterial genes whose products appear to be responsible for this process. In general, transformation is a complex, energy-requiring developmental process. In order for a bacterium to bind, take up and recombine exogenous DNA into its chromosome, it must become competent, that is, enter a special physiological state. For example, competence development in Bacillus subtilis requires expression of about 40 genes. The DNA integrated into the host chromosome is usually (but with rare exceptions) derived from another bacterium of the same species, and is thus homologous to the resident chromosome.

The term “administer,” “administering,” or “administration” refers to implanting, absorbing, ingesting, injecting, inhaling, or otherwise introducing a compound described herein, or a composition thereof, in or on a subject.

The terms “treatment,” “treat,” and “treating” refer to reversing, alleviating, delaying the onset of, or inhibiting the progress of a disease described herein. In some embodiments, treatment may be administered after one or more signs or symptoms of the disease have developed or have been observed. In other embodiments, treatment may be administered in the absence of signs or symptoms of the disease. For example, treatment may be administered to a susceptible subject prior to the onset of symptoms (e.g., in light of a history of symptoms and/or in light of exposure to a pathogen). Treatment may also be continued after symptoms have resolved, for example, to delay or prevent recurrence.

The terms “condition,” “disease,” and “disorder” are used interchangeably.

An “effective amount” of a compound described herein refers to an amount sufficient to elicit the desired biological response. An effective amount of a compound described herein may vary depending on such factors as the desired biological endpoint, the pharmacokinetics of the compound, the condition being treated, the mode of administration, and the age and health of the subject. In certain embodiments, an effective amount is a therapeutically effective amount. In certain embodiments, an effective amount is a prophylactic treatment. In certain embodiments, an effective amount is the amount of a compound described herein in a single dose. In certain embodiments, an effective amount is the combined amounts of a compound described herein in multiple doses.

A “therapeutically effective amount” of a compound described herein is an amount sufficient to provide a therapeutic benefit in the treatment of a condition or to delay or minimize one or more symptoms associated with the condition. A therapeutically effective amount of a compound means an amount of therapeutic agent, alone or in combination with other therapies, which provides a therapeutic benefit in the treatment of the condition. The term “therapeutically effective amount” can encompass an amount that improves overall therapy, reduces or avoids symptoms, signs, or causes of the condition, and/or enhances the therapeutic efficacy of another therapeutic agent. In certain embodiments, a therapeutically effective amount is an amount sufficient for inhibiting microbial virulence. In certain embodiments, a therapeutically effective amount is an amount sufficient for treating an infection. In certain embodiments, a therapeutically effective amount is an amount sufficient for inhibiting microbial virulence and treating an infection.

A “prophylactically effective amount” of a compound described herein is an amount sufficient to prevent a condition, or one or more symptoms associated with the condition or prevent its recurrence. A prophylactically effective amount of a compound means an amount of a therapeutic agent, alone or in combination with other agents, which provides a prophylactic benefit in the prevention of the condition. The term “prophylactically effective amount” can encompass an amount that improves overall prophylaxis or enhances the prophylactic efficacy of another prophylactic agent. In certain embodiments, a prophylactically effective amount is an amount sufficient for inhibiting microbial virulence. In certain embodiments, a prophylactically effective amount is an amount sufficient for treating an infection. In certain embodiments, a prophylactically effective amount is an amount sufficient for inhibiting microbial virulence and treating an infection.

As used herein, use of the phrase “at least one instance” refers to 1, 2, 3, 4, or more instances, but also encompasses a range, e.g., for example, from 1 to 4, from 1 to 3, from 1 to 2, from 2 to 4, from 2 to 3, or from 3 to 4 instances, inclusive.

These and other exemplary substituents are described in more detail in the Detailed Description, Examples, and Claims. The invention is not intended to be limited in any manner by the above exemplary listing of substituents.

### BRIEF DESCRIPTION OF THE DRAWINGS

The accompanying drawings, which constitute a part of this specification, illustrate several embodiments of the invention and together with the description, serve to explain the principles of the invention.

FIGS. 1A and 1B are ¹H NMR spectra of carboxymethyl cellulose (FIG. 1A) and carboxymethyl cellulose glycosylated with glucose (FIG. 1B).

FIG. 2 is a bar graph showing the inhibitory effects against biofilm formation by S. mutans using exemplary polymers, glucose-CMC, galactose-CMC, fucose-CMC, and mannose-CMC (prepared from 90 kDa CMC), in a biofilm inhibition assay. Non-functionalized CMC was included as a control.

FIG. 3 is a bar graph showing the inhibitory effects against biofilm formation by S. mutans using exemplary polymer glucose-CMC (prepared from 90 kDa CMC) in comparison to non-functionalized 90 kDa CMC mixed with unbound glucose (1% and 0.2% (w/v)) in a biofilm inhibition assay. Non-functionalized CMC was included as a control.

FIG. 4 is a graph showing the growth rate of S. mutans in media only (25% Todd Hewitt Broth (TH)+1% sucrose (w/v)) and media mixed with exemplary polymer glucose-CMC prepared from 250 kDa CMC.

FIG. 5 is a bar graph showing the inhibitory effects against biofilm formation by S. mutans using exemplary polymer glucose-CMC (prepared from 250 kDa CMC) in comparison to media only.

FIG. 6 is a bar graph showing the inhibition of S. mutans natural transformation by exemplary polymer glucose-CMC and non-functionalized CMC in a horizontal gene transfer assay. Media (25% TH+1% sucrose (w/v)) was included as a control.

### DETAILED DESCRIPTION OF CERTAIN EMBODIMENTS

Provided herein are polymers that are synthetic mucins, methods of preparing such polymers, and methods of using the polymers. The polymers comprise a carboxymethyl cellulose backbone substituted with monosaccharide (e.g., glucose), oligosaccharide (e.g., mucin glycan), and/or polysaccharide sidechains, and are useful as pharmaceutical compositions and prebiotic compositions among other uses. The structure of the polymers confer certain unexpected and advantageous properties on the polymers. For example, the polymers may inhibit microbial virulence and/or biofilm formation. This activity is useful for preventing the growth of and/or killing microbes, and preventing the exchange of genetic material between microbes, which often leads to the acquisition of novel virulence and antibiotic resistance traits. The ability of the inventive polymers to inhibit virulence traits such as natural transformation is surprising and unexpected, as this property was previously identified to be unique to the naturally occurring salivary mucin MUC5B.

### Polymers

The design of the synthetic mucins of the present disclosure is based on the following criteria: the polymer backbone should be sufficiently long to mimic the protein backbone length of mucins; the glycosylation method should be covalent and not susceptible to enzymatic degradation; the glycosylation should be uniform; and the final glycopolymer should have a net negative charge to mimic the charge of mucins. The polymers disclosed herein may have one or more of these design elements.

To satisfy the above criteria, carboxymethyl cellulose (CMC) was employed as the polymer backbone. CMC is relatively inert when tested for activity against microbes. The carboxylation of the backbone provides a net negative charge that is similar to intact mucins, and the polymer is commercially available in various lengths (30 kDa to >700 kDa depending on the cellulose source). The CMC may be glycosylated via an amide forming reaction between a glycosylamine and one or more carboxyl groups of the CMC to form polymers of the present disclosure. For each commercially available molecular weight of CMC, the degree of carboxylation ranges from 0.7 to 1.2 per glucose monomer, which allows for tunability with respect to both the hydrophilicity as well as degree of glycosylation.

Accordingly, the polymers provided herein comprise a backbone of repeating glucopyranose monomer units (“backbone units”). The polymers are of Formula (I):

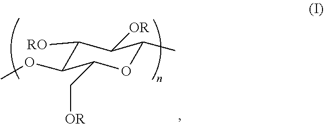

or salts thereof, wherein:

each occurrence of R is independently hydrogen, —CH₂C(O)OH, —CH₂C(O)OR¹, —CH₂C(O)NHR¹, or R¹;

each occurrence of R¹ is independently a monosaccharide, oligosaccharide, or polysaccharide; and

n is equal to or greater than 20;

wherein the polymer has, on average, about 0.5 to about 2.0 R groups in each repeat unit that are —CH₂C(O)OH, —CH₂C(O)OR¹, or —CH₂C(O)NHR¹.

In certain embodiments, n is an integer between 20 and 100000, inclusive. In certain embodiments, n is an integer between 20 and 10000, inclusive. In certain embodiments, n is an integer is between 20 and 1000, inclusive. In certain embodiments, n is an integer is between 20 and 1000, inclusive. In certain embodiments, n is an integer is between 50 and 1000, inclusive. In certain embodiments, n is an integer between 20 and 100, inclusive. In certain embodiments, n is an integer between 50 and 100, inclusive. In certain embodiments, n is an integer between 100 and 10000, inclusive. In certain embodiments, n is an integer between 1000 and 10000, inclusive. In certain embodiments, n is at least 20, 30, 40, 50, 100, 500, 1000, 2000, 3000, 5000, 10000, at least 20000, at least 50000, or at least 100000. In certain embodiments, n is at least 20. In certain embodiments, n is at least 50.

The polymers may be of any molecular weight. In certain embodiments, the polymer has a molecular weight of up to 1,000,000 Da, up to 900,000 Da, up to 800,000 Da, up to 700,000 Da, up to 600,000 Da, up to 500,000 Da, up to 400,000 Da, up to 300,000 Da, up to 200,000 Da, up to 100,000 Da, up to 90,000 Da, up to 80,000 Da, up to 70,000 Da, up to 60,000 Da, up to 50,000 Da, up to 40,000 Da, up to 30,000 Da, up to 20,000 Da, up to 10,000 Da, up to 5,000 Da, up to 4,000 Da, up to 3,000 Da, up to 2,000 Da, or up to 1,000 Da. In certain embodiments, the polymer has a molecular weight of at least 1,000 Da, at least 2,000 Da, at least 3,000 Da, at least 4,000 Da, at least 5,000 Da, at least 10,000 Da, at least 20,000 Da, at least 30,000 Da, at least 40,000 Da, at least 50,000 Da, at least 60,000 Da, at least 70,000 Da, at least 80,000 Da, at least 90,000 Da, at least 100,000 Da, at least 200,000 Da, at least 300,000 Da, at least 400,000 Da, at least 500,000 Da, at least 600,000 Da, at least 700,000 Da, at least 800,000 Da, at least 900,000 Da, or at least 1,000,000 Da. In certain embodiments, the molecular weight is an amount between a molecular weight described in this paragraph and another molecular weight in this paragraph, inclusive.

In certain embodiments, the polymer has a molecular weight of about 10,000 Da to about 500,000 Da, about 10,000 Da to about 400,000 Da, about 10,000 Da to about 300,000 Da, about 10,000 Da to about 200,000 Da, about 10,000 Da to about 100,000 Da, about 100,000 Da to about 1,000,000 Da, about 100,000 Da to about 500,000 Da, about 100,000 Da to about 400,000 Da, or about 100,000 Da to about 300,000 Da. In certain embodiments, the molecular weight is determined by size exclusion chromatography.

In certain embodiments, the polymer comprises carboxylic acid moieties that are deprotonated and carry a negative charge at physiological pH (e.g., 7.4). In certain embodiments, the polymers have a net negative charge at a pH of about 3, about 4, about 5, about 6, about 6.5, about 6.6, about 6.7, about 6.8, about 6.9, about 7, about 7.1, about 7.2, about 7.3, about 7.4, about 7.5, about 7.6, about 7.7, about 7.8, about 7.9, about 8, about 9, about 10, about 11, about 12, about 13, or about 14. In certain embodiments, the polymers have a net negative charge at a pH of about 7.4. In certain embodiments, the polymers have a net negative charge at physiological pH.

In certain embodiments, the unsubstituted carboxymethyl cellulose polymer has a charge of about −0.5 to about −2.0 per monomer. In certain embodiments, the unsubstituted carboxymethyl cellulose polymer has a charge of about −0.5, about −0.6, about −0.7, about −0.8, about −0.9, about −1.0, about −1.1, about −1.2, about −1.3, about −1.4, about −1.5, about −1.6, about −1.7, about −1.8, about −1.9, or about −2.0. In certain embodiments, the unsubstituted carboxymethyl cellulose polymer has a charge of about −0.7 to about −1.2.

### Group R¹

In certain embodiments, R¹ is a monosaccharide, oligosaccharide, or polysaccharide. In certain embodiments, R¹ is a monosaccharide or oligosaccharide. In certain embodiments, R¹ is a monosaccharide. In certain embodiments, R¹ is a disaccharide. In certain embodiments, R¹ is a trisaccharide. In certain embodiments, R¹ is an oligosaccharide. In certain embodiments, R¹ is a polysaccharide.

In certain embodiments, R¹ is a monosaccharide. In certain embodiments, R¹ is glucose, galactose, mannose, fucose, sialic acid, glucosamine, galactosamine, mannosamine, fucosamine, N-acetylglucosamine, or N-acetylgalactosamine. In certain embodiments, R¹ is glucose, galactose, mannose, or fucose. In certain embodiments, R¹ is glucose, galactose, or fucose. In certain embodiments, R¹ is glucose or galactose. In certain embodiments, R¹ is glucose. In certain embodiments, R¹ is galactose. In certain embodiments, R¹ is mannose or fucose. In certain embodiments, R¹ is mannose. In certain embodiments, R¹ is fucose.

In certain embodiments, R¹ is an oligosaccharide. In certain embodiments, the oligosaccharide is a fructooligosaccharide, a galactooligosaccharide, a mannan oligosaccharide, raffinose, lactose, a human milk oligosaccharide, or a mucin glycan. In certain embodiments, R¹ is lactose, a human milk oligosaccharide, or a mucin glycan. In certain embodiments, R¹ is a human milk oligosaccharide or a mucin glycan.

In certain embodiments, R¹ is a mucin glycan. Mucin glycans are known oligosaccharides which are found on the protein backbone of mucins. In certain embodiments, the mucin glycan is of the formula:

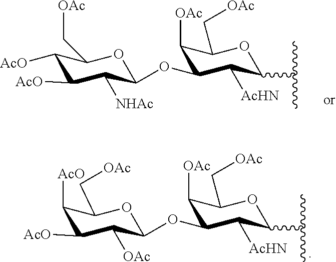

The known mucin glycans described above may be prepared and coupled to CMC by glycosylation methods known to one of ordinary skill in the art.

In certain embodiments, R¹ is a human milk oligosaccharide. Human milk oligosaccharides are complex glycans found in human breast milk. These glycans are not metabolized by the infant and instead are processed by various microbes within the gastrointestinal tract. Human milk oligosaccharides have multi-faceted functions. From controlling the immune response in the bloodstream to acting as decoy binding sites for pathogenic microbes, human milk oligosaccharides are key regulators of infant health and help establish the infant microbiota.

Structurally, human milk oligosaccharides have similarities with mucin glycans. Human milk oligosaccharides have lactose as the core sugar while mucins have GalNac attached to a serine or threonine. The terminal structures of the glycans are highly similar, with branched galactose and GlcNac chains terminated with either sialic acid or fucose. Although the genetic and enzymatic regulation of Human milk oligosaccharide production is not well-defined, the monosaccharides used in the synthesis pathways are the same for mucin glycans. Since these processes are stochastic, within a healthy mother's breast milk, over 100 glycan varieties have been identified. The nine most common human milk oligosaccharides were found to have considerable structural similarity with glycans previously identified on the mucin, Mucin 2 (MUC2). These human milk oligosaccharides include lacto-N-tetraose, lacto-N-fucopentaose I (LNFP I), lacto-N-fecopentaose II (LNFP II), lacto-N-fucopentaose III (LNFP III), lacto-N-difucohexaose I, NeuAc(alpha2-6)lactose, NeuAc(alpha2-3)lactose, NeuAc-lacto-N-tetraose a, NeuAc-lacto-N-tetraose b, and NeuAc2-lacto-N-tetraose.

In certain embodiments, R¹ is lacto-N-tetraose, lacto-N-fucopentaose I (LNFP I), lacto-N-fecopentaose II (LNFP II), lacto-N-fucopentaose III (LNFP III), lacto-N-difucohexaose I, NeuAc(alpha2-6)lactose, NeuAc(alpha2-3)lactose, NeuAc-lacto-N-tetraose a, NeuAc-lacto-N-tetraose b, NeuAc2-lacto-N-tetraose, or a combination thereof.

In certain embodiments, the molecular weight of the R¹ groups comprise, by weight, about 1% to about 50%, about 1% to about 40%, about 1% to about 30%, about 1% to about 20%, about 1% to about 15%, about 1% to about 10%, about 5% to about 50%, about 5% to about 40%, about 5% to about 30%, about 5% to about 20%, about 5% to about 15%, about 5% to about 10%, about 8% to about 12%, or about 9% to about 11% of the molecular weight of the polymer. In certain embodiments, the molecular weight of the R¹ groups comprise, by weight, about 1%, about 2%, about 3%, about 4%, about 5%, about 10%, about 15%, about 20%, about 25%, about 30%, about 40%, or about 50% of the molecular weight of the polymer.

In certain embodiments, at least 1% of the carboxyl groups of the polymer are glycosylated with an R¹ group (e.g., monosaccharide, oligosaccharide, and/or polysaccharide). In certain embodiments, at least 10% of the carboxyl groups of the polymer are glycosylated with an R¹ group (e.g., monosaccharide, oligosaccharide, and/or polysaccharide). In certain embodiments, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 99% of the carboxyl groups of the polymer are glycosylated with an R¹ group (e.g., monosaccharide, oligosaccharide, and/or polysaccharide).

### Group R

The carboxymethyl cellulose backbone of the polymer comprises individual repeat units of glucose, any of which may have a carboxymethyl group or derivative of a carboxymethyl group (—CH₂C(O)OH, —CH₂C(O)OR¹, —CH₂C(O)NHR¹) as one or more R groups. The distribution of the carboxymethyl group or derivative of the carboxymethyl group throughout the polymer and in each repeat unit is determined by the degree of carboxylation of the unsubstituted carboxymethyl cellulose polymer. The degree of carboxylation is the number of carboxymethyl groups, on average, in each repeat unit. In certain embodiments, the degree of carboxylation of the unsubstituted carboxymethyl cellulose polymer is about 0.5 to about 2.0. In certain embodiments, the degree of carboxylation of the unsubstituted carboxymethyl cellulose polymer is about 0.5, about 0.6, about 0.7, about 0.8, about 0.9, about 1.0, about 1.1, about 1.2, about 1.3, about 1.4, about 1.5, about 1.6, about 1.7, about 1.8, about 1.9, or about 2.0. In certain embodiments, the degree of carboxylation of the unsubstituted carboxymethyl cellulose polymer is about 0.7 to about 1.2. In certain embodiments, the unsubstituted carboxymethyl cellulose polymer is glycosylated via an amide forming reaction between one or more glycosylamines and one or more carboxyl groups of the unsubstituted carboxymethyl cellulose to form polymers of the present disclosure.

In certain embodiments, each occurrence of R is independently hydrogen, —CH₂C(O)OH, —CH₂C(O)OR¹, —CH₂C(O)NHR¹, or R¹, wherein the polymer has, on average, about 0.5 to about 2.0 R groups in each repeat unit that are carboxymethyl or a derivative of a carboxymethyl group (i.e., —CH₂C(O)OH, —CH₂C(O)OR¹, or —CH₂C(O)NHR¹). In certain embodiments, each occurrence of R is independently hydrogen, —CH₂C(O)OH, —CH₂C(O)OR¹, or —CH₂C(O)NHR¹. In certain embodiments, each occurrence of R is independently hydrogen, —CH₂C(O)OH, or —CH₂C(O)NHR¹.

In certain embodiments, each occurrence of R is independently hydrogen, —CH₂C(O)OH, —CH₂C(O)OR¹, or —CH₂C(O)NHR¹; and R¹ is a monosaccharide. In certain embodiments, each occurrence of R is independently hydrogen, —CH₂C(O)OH, —CH₂C(O)OR¹, or —CH₂C(O)NHR¹; and R¹ is an oligosaccharide (e.g., human milk oligosaccharide, mucin glycan). In certain embodiments, each occurrence of R is independently hydrogen, —CH₂C(O)OH, or —CH₂C(O)NHR¹; and R¹ is a monosaccharide. In certain embodiments, each occurrence of R is independently hydrogen, —CH₂C(O)OH, or —CH₂C(O)NHR¹; and R¹ is an oligosaccharide (e.g., human milk oligosaccharide, mucin glycan).

In certain embodiments, R is hydrogen, —CH₂C(O)OH,

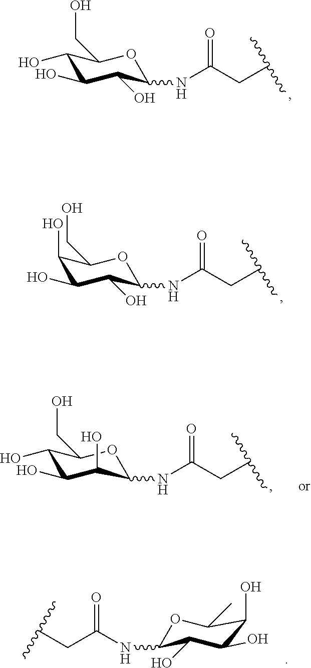

Given that the degree of carboxylation is an average of carboxymethyl groups per repeat unit of CMC, there may be a range of structural diversity in the different repeat units of any polymer of the present disclosure. The distribution of the carboxymethyl group or derivative of the carboxymethyl group in each repeat unit is such that they typically reside on the C-6 R group. However, the degree of carboxylation may be greater than 1, and the C-2 and/or C-3 R group may be a carboxymethyl group or derivative of a carboxymethyl in some repeat units.

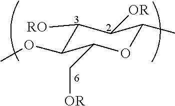

In certain repeat units of the polymer, the C-6 R group is —CH₂C(O)OH, —CH₂C(O)OR¹, or —CH₂C(O)NHR¹, and the C-2 and C-3 R groups are hydrogen. In certain repeat units of the polymer, the C-6 and C-2 R groups are hydrogen, and the C-3 R group is —CH₂C(O)OH, —CH₂C(O)OR¹, or —CH₂C(O)NHR¹. In certain repeat units of the polymer, the C-6 and C-3 R groups are hydrogen, and the C-2 R group is —CH₂C(O)OH, —CH₂C(O)OR¹, or —CH₂C(O)NHR¹. In certain repeat units of the polymer, the C-6 and the C-2 R group are independently —CH₂C(O)OH, —CH₂C(O)OR¹, or —CH₂C(O)NHR¹, and the C-3 R group is hydrogen. In certain repeat units of the polymer, the C-6 and the C-3 R group are independently —CH₂C(O)OH, —CH₂C(O)OR¹, or —CH₂C(O)NHR¹, and the C-3 R group is hydrogen. In certain repeat units of the polymer, the C-2 and the C-3 R group are independently —CH₂C(O)OH, —CH₂C(O)OR¹, or —CH₂C(O)NHR¹, and the C-6 R group is hydrogen. In certain repeat units of the polymer, the C-2, C-3, and C-6 R groups are independently —CH₂C(O)OH, —CH₂C(O)OR¹, or —CH₂C(O)NHR¹. In certain repeat units of the polymer, the C-2, C-3, and C-6 R groups are hydrogen.

In certain repeat units of the polymer, the C-6 R group is —CH2C(O)OH or —CH₂C(O)NHR¹, and the C-2 and C-3 R groups are hydrogen. In certain repeat units of the polymer, the C-6 and C-2 R groups are hydrogen, and the C-3 R group is —CH₂C(O)OH or —CH₂C(O)NHR¹. In certain repeat units of the polymer, the C-6 and C-3 R groups are hydrogen, and the C-2 R group is —CH₂C(O)OH or —CH₂C(O)NHR¹. In certain repeat units of the polymer, the C-6 and the C-2 R group are independently —CH₂C(O)OH or —CH₂C(O)NHR¹, and the C-3 R group is hydrogen. In certain repeat units of the polymer, the C-6 and the C-3 R group are independently —CH₂C(O)OH or —CH₂C(O)NHR¹, and the C-3 R group is hydrogen. In certain repeat units of the polymer, the C-2 and the C-3 R group are independently —CH₂C(O)OH or —CH₂C(O)NHR¹, and the C-6 R group is hydrogen. In certain repeat units of the polymer, the C-2, C-3, and C-6 R groups are independently —CH₂C(O)OH or —CH₂C(O)NHR¹.

In certain repeat units of the polymer, the C-6 R group is CH2C(O)NHR¹, and the C-2 and C-3 R groups are hydrogen. In certain repeat units of the polymer, the C-6 and C-2 R groups are hydrogen, and the C-3 R group is —CH₂C(O)NHR¹. In certain repeat units of the polymer, the C-6 and C-3 R groups are hydrogen, and the C-2 R group is —CH₂C(O)NHR¹. In certain repeat units of the polymer, the C-6 and the C-2 R group are —CH₂C(O)NHR¹, and the C-3 R group is hydrogen. In certain repeat units of the polymer, the C-6 and the C-3 R group are —CH₂C(O)NHR¹, and the C-3 R group is hydrogen. In certain repeat units of the polymer, the C-2 and the C-3 R group are —CH₂C(O)NHR¹, and the C-6 R group is hydrogen. In certain repeat units of the polymer, the C-2, C-3, and C-6 R groups are —CH₂C(O)NHR¹.

In certain repeat units of the polymer, the C-6 R group is —CH₂C(O)OH, and the C-2 and C-3 R groups are hydrogen. In certain repeat units of the polymer, the C-6 and C-2 R groups are hydrogen, and the C-3 R group is —CH₂C(O)OH. In certain repeat units of the polymer, the C-6 and C-3 R groups are hydrogen, and the C-2 R group is —CH₂C(O)OH. In certain repeat units of the polymer, the C-6 and the C-2 R group are —CH₂C(O)OH, and the C-3 R group is hydrogen. In certain repeat units of the polymer, the C-6 and the C-3 R group are —CH₂C(O)OH, and the C-3 R group is hydrogen. In certain repeat units of the polymer, the C-2 and the C-3 R group are —CH₂C(O)OH, and the C-6 R group is hydrogen. In certain repeat units of the polymer, the C-2, C-3, and C-6 R groups are —CH₂C(O)OH.

In certain embodiments, the polymer has, on average, at least 0.5, at least 0.6, at least 0.7, at least 0.8, at least 0.9, at least 1.0, at least 1.1, at least 1.2, at least 1.3, at least 1.4, at least 1.5, at least 1.6, at least 1.7, at least 1.8, at least 1.9, or at least 2.0 R groups in each repeat unit that are —CH₂C(O)OH, —CH₂C(O)OR¹, or —CH₂C(O)NHR¹. In certain embodiments, the polymer has, on average, about 0.5 to about 2.0, about 0.5 to about 1.9, about 0.5 to about 1.8, about 0.5 to about 1.7, about 0.5 to about 1.6, about 0.5 to about 1.5, about 0.5 to about 1.4, about 0.5 to about 1.3, about 0.5 to about 1.2, about 0.6 to about 1.2, about 0.7 to about 1.2, about 0.8 to about 1.2, about 0.9 to about 1.2, about 1.0 to about 1.2, about 1.1 to about 1.2, about 0.9 to about 1.1, about 0.9 to about 1.0, or about 1.0 to about 1.1 R groups that are —CH₂C(O)OH, —CH₂C(O)OR¹, or —CH₂C(O)NHR¹. In certain embodiments, the polymer has, on average, about 0.5, about 0.6, about 0.7, about 0.8, about 0.9, about 1.0, about 1.1, about 1.2, about 1.3, about 1.4, about 1.5, about 1.6, about 1.7, about 1.8, about 1.9, or about 2.0 R groups in each repeat unit that are —CH₂C(O)OH, —CH₂C(O)OR¹, or —CH₂C(O)NHR¹.

In certain embodiments, the polymer has, on average, at least 0.5, at least 0.6, at least 0.7, at least 0.8, at least 0.9, at least 1.0, at least 1.1, at least 1.2, at least 1.3, at least 1.4, at least 1.5, at least 1.6, at least 1.7, at least 1.8, at least 1.9, or at least 2.0 R groups in each repeat unit that are —CH₂C(O)OH or —CH₂C(O)NHR¹. In certain embodiments, the polymer has, on average, about 0.5 to about 2.0, about 0.5 to about 1.9, about 0.5 to about 1.8, about 0.5 to about 1.7, about 0.5 to about 1.6, about 0.5 to about 1.5, about 0.5 to about 1.4, about 0.5 to about 1.3, about 0.5 to about 1.2, about 0.6 to about 1.2, about 0.7 to about 1.2, about 0.8 to about 1.2, about 0.9 to about 1.2, about 1.0 to about 1.2, about 1.1 to about 1.2, about 0.9 to about 1.1, about 0.9 to about 1.0, or about 1.0 to about 1.1 R groups that are —CH₂C(O)OH or —CH₂C(O)NHR¹. In certain embodiments, the polymer has, on average, about 0.5, about 0.6, about 0.7, about 0.8, about 0.9, about 1.0, about 1.1, about 1.2, about 1.3, about 1.4, about 1.5, about 1.6, about 1.7, about 1.8, about 1.9, or about 2.0 R groups in each repeat unit that are —CH₂C(O)OH or —CH₂C(O)NHR¹.

In certain embodiments, when R is —CH₂C(O)OH, the carboxy group may be a salt form. In certain embodiments, the salt may include a group I or group II metal ion (e.g., lithium, sodium, potassium, rubidium, cesium, beryllium, magnesium, calcium, strontium, barium).

### End Groups

In certain embodiments, the polymer comprises end groups. In certain embodiments, the end groups are hydrogen, hydroxyl, substituted or unsubstituted alkyl, or carboxyl. In certain embodiments, the end groups are hydrogen, hydroxyl, methyl, or COOH.

### Methods for Preparing the Polymers

The present disclosure also provides methods for preparing the polymers described herein. The methods comprise glycosylating carboxymethyl cellulose with one or more monosaccharide, oligosaccharide, or polysaccharide. Any suitable glycosylation reactions known in the art may be employed. Specifically, the carboxyl groups of carboxymethyl cellulose may provide glycosylation sites through amide forming reactions with glycosylamines. The glycosylamines may be formed by converting the aldehyde/acetal groups of reducing sugars to amino groups by the Kotchetkov reaction as illustrated in Scheme 1 (below).

Accordingly, provided herein is a method of producing a polymer described herein, the method comprising the step of coupling carboxymethyl cellulose with one or more monosaccharide, oligosaccharide, or polysaccharide.

In certain embodiments, the method comprises amination of the one or more monosaccharide, oligosaccharide, or polysaccharide prior to coupling carboxymethyl cellulose with the one or more monosaccharide, oligosaccharide, or polysaccharide to form a glycosylamine. In certain embodiments, the amination of the one or more monosaccharide, oligosaccharide, or polysaccharide is amination of the reducing end of the one or more monosaccharide, oligosaccharide, or polysaccharide (e.g., employing the Kotchekov reaction). In certain embodiments, the amination is achieved by reaction of a monosaccharide, oligosaccharide, or polysaccharide with ammonium bicarbonate. In certain embodiments, up to about 10, about 20, about 30, about 40, or about 50 equivalents (w/w) of ammonium bicarbonate may be used in the amination reaction. The amination may be achieved at room temperature.

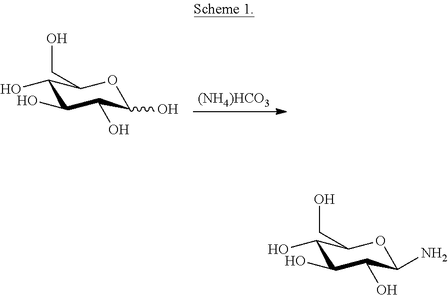

In certain embodiments, the coupling of carboxymethyl cellulose with one or more monosaccharide, oligosaccharide, or polysaccharide is achieved through an amide bond formation with the carboxyl groups of the carboxymethyl cellulose (e.g., with a glycosylamine). In certain embodiments, the amide bond formation is promoted by amide coupling reagents (e.g., 1-ethyl-3-(3-dimethylaminopropyl)carbodiimide (EDC), hydroxybenzotriazole (HOB t)). In certain embodiments, the amide coupling reagents (e.g., EDC, HOBt) are reacted with carboxymethyl cellulose. In certain embodiments, the amide coupling reagents (e.g., EDC, HOBt) are reacted with carboxymethyl cellulose prior to reacting with a glycosylamine. In certain embodiments, each amide coupling reagent (e.g., EDC, HOBt) is reacted with carboxymethyl cellulose in a 1:1 molar ratio of amide coupling reagent:each carboxyl group in the polymer (e.g., 1 equivalent of EDC and/or 1 equivalent of HOBt for each carboxyl group in CMC). In certain embodiments, the glycosylamine is then added to the reaction mixture in about a 1:1, 1.1:1, 1.2:1, 1.3:1, 1.4:1, 1.5:1, 1.6:1, 1.7:1, 1.8:1, 1.9:1, 2:1, 2.1:1, 2.2:1, 2.3:1, 2.4:1, 2.5:1, 2.6:1, 2.7:1, 2.8:1, 2.9:1, or 3:1 molar ratio (glycosylamine:each carboxyl group). For example, in one embodiment of a repeat unit of the polymer, coupling may be achieved as illustrated in Scheme 2 (below), wherein R¹ is as defined herein.

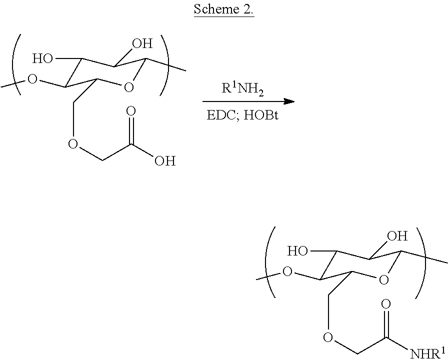

### Compositions and Administration

The present disclosure provides a composition comprising a polymer of Formula (I), or a salt thereof, and optionally an acceptable carrier. In embodiments, the composition is a pharmaceutical composition, a prebiotic composition, a nutraceutical composition, or a food product.

In certain embodiments, the composition is a pharmaceutical composition comprising a polymer of Formula (I), or a salt thereof, and a pharmaceutically acceptable carrier. In certain embodiments, the polymer of Formula (I) is provided in an effective amount in the composition. In certain embodiments, the effective amount is a therapeutically effective amount. In certain embodiments, the effective amount is a prophylactically effective amount. In certain embodiments, the effective amount is an amount effective for treating an infection (e.g., bacterial, protozoan, fungal) in a subject in need thereof. In certain embodiments, the effective amount is an amount effective for preventing an infection (e.g., bacterial, protozoan, fungal) in a subject in need thereof. In certain embodiments, the effective amount is an amount effective for reducing the risk of developing an infection (e.g., bacterial, protozoan, fungal) in a subject in need thereof. In certain embodiments, the effective amount is an amount effective for inhibiting the virulence of a microbe (e.g., bacteria, archaea, protozoa, fungi, algae).

In certain embodiments, the effective amount is an amount effective for inhibiting the formation of a biofilm on a surface. In certain embodiments, the effective amount is an amount effective for preventing the formation of a biofilm on a surface. In certain embodiments, the effective amount is an amount effective for inhibiting or preventing the formation of a biofilm on a surface, wherein the biofilm comprises bacteria, archaea, protozoa, fungi, and/or algae.

In certain embodiments, the surface being treated is a surface in or on a subject (e.g., tooth, wound). In certain embodiments, the surface being treated is a surface on an inanimate object (e.g., medical device).

In certain embodiments, the subject is an animal. The animal may be of either sex and may be at any stage of development. In certain embodiments, the subject described herein is a human. In certain embodiments, the subject is a non-human animal. In certain embodiments, the subject is a mammal. In certain embodiments, the subject is a non-human mammal. In certain embodiments, the subject is a domesticated animal, such as a dog, cat, cow, pig, horse, sheep, or goat. In certain embodiments, the subject is a companion animal, such as a dog or cat. In certain embodiments, the subject is a livestock animal, such as a cow, pig, horse, sheep, or goat. In certain embodiments, the subject is a zoo animal. In another embodiment, the subject is a research animal, such as a rodent (e.g., mouse, rat), dog, pig, or non-human primate. In certain embodiments, the animal is a genetically engineered animal. In certain embodiments, the animal is a transgenic animal (e.g., transgenic mice and transgenic pigs). In certain embodiments, the subject is a fish or reptile.

In certain embodiments, the effective amount is an amount effective for inhibiting biofilm formation on a surface by at least about 10%, at least about 20%, at least about 30%, at least about 40%, at least about 50%, at least about 60%, at least about 70%, at least about 80%, at least about 90%, at least about 95%, at least about 98%, or at least about 99% as compared to a surface untreated with a polymer of Formula (I). In certain embodiments, the effective amount is an amount effective for inhibiting biofilm formation on a surface by a range between a percentage described in this paragraph and another percentage described in this paragraph, inclusive. In certain embodiments, the biofilm comprises bacteria, archaea, protozoa, fungi, and/or algae.

The present disclosure provides pharmaceutical compositions comprising a polymer of Formula (I) for use in treating an infection in a subject in need thereof. In certain embodiments, the composition is for use in treating a protozoan infection (e.g., infection(s) caused by Entamoeba histolytica, Plasmodium, Giardia lamblia, and/or Trypanosoma brucei). In certain embodiments, the composition is for use in treating a fungal infection (e.g., infection(s) caused by Aspergillus, Blastomyces, Candida (e.g., Candida albicans), Coccidioides, Cryptococcus (e.g., Cryptococcus gattii, Cryptococcus neoformans), Histoplasma, Pneumocystis jirovecii, Sporothrix, Exserohilum and/or Cladosporium). In certain embodiments, the composition is for use in treating a bacterial infection (e.g., infection(s) caused by Actinomyces, Bacillus, Becteroides, Bordatella, Borrelia, Brucella, Camylobacter, Chlamydia, Chlamydophila, Clostridium, Corynebacterium, Ehrlichia, Enterococcus, Escherichia, Francisella, Haemophilus, Helicobacter, Klebsiella, Legionella, Leptospira, Listeria, Mycobacterium, Mycoplasma, Neisseria, Pseudomonas (e.g., Pseudomonas aeruginosa), Nocardia, Rickettsia, Salmonella, Shigella, Staphylococcus (e.g., Staphylococcus aureus), Streptococcus (e.g., Streptococcus mutans), Treponema, Vibrio, and/or Yersinia). In certain embodiments, the composition is for use in treating infection caused by Streptococcus mutans, Pseudomonas aeruginosa, Staphylococcus aureus, or Candida albicans. In certain embodiments, the composition is for use in treating infection caused by Streptococcus mutans.

Pharmaceutical compositions described herein can be prepared by any method known in the art of pharmacology. In general, such preparatory methods include the steps of bringing the composition comprising a polymer of Formula (I) into association with a carrier and/or one or more other accessory ingredients, and then, if necessary and/or desirable, shaping and/or packaging the product into a desired single- or multi-dose unit.

Pharmaceutical compositions can be prepared, packaged, and/or sold in bulk, as a single unit dose, and/or as a plurality of single unit doses. As used herein, a “unit dose” is a discrete amount of the pharmaceutical composition comprising a predetermined amount of the active ingredient. The amount of the active ingredient is generally equal to the dosage of the active ingredient which would be administered to a subject and/or a convenient fraction of such a dosage, such as, for example, one-half or one-third of such a dosage.

Relative amounts of the active ingredient, the pharmaceutically acceptable carrier, and/or any additional ingredients in a pharmaceutical composition of the invention will vary, depending upon the identity, size, and/or condition of the subject treated and further depending upon the route by which the composition is to be administered. By way of example, the composition may comprise between 0.1% and 100% (w/w) active ingredient.

The pharmaceutically acceptable carrier comprises one or more pharmaceutically acceptable excipients. Pharmaceutically acceptable excipients used in the manufacture of provided pharmaceutical compositions include inert diluents, dispersing and/or granulating agents, surface active agents and/or emulsifiers, disintegrating agents, binding agents, preservatives, buffering agents, lubricating agents, and/or oils. Excipients such as cocoa butter and suppository waxes, coloring agents, coating agents, sweetening, flavoring, and perfuming agents may also be present in the composition.

Exemplary diluents include calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, and mixtures thereof.

Exemplary granulating and/or dispersing agents include potato starch, corn starch, tapioca starch, sodium starch glycolate, clays, alginic acid, guar gum, citrus pulp, agar, bentonite, cellulose, and wood products, natural sponge, cation-exchange resins, calcium carbonate, silicates, sodium carbonate, cross-linked poly(vinyl-pyrrolidone) (crospovidone), sodium carboxymethyl starch (sodium starch glycolate), carboxymethyl cellulose, cross-linked sodium carboxymethyl cellulose (croscarmellose), methylcellulose, pregelatinized starch (starch 1500), microcrystalline starch, water insoluble starch, calcium carboxymethyl cellulose, magnesium aluminum silicate (Veegum), sodium lauryl sulfate, quaternary ammonium compounds, and mixtures thereof.

Exemplary surface active agents and/or emulsifiers include natural emulsifiers (e.g. acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g. bentonite (aluminum silicate) and Veegum (magnesium aluminum silicate)), long chain amino acid derivatives, high molecular weight alcohols (e.g. stearyl alcohol, cetyl alcohol, oleyl alcohol, triacetin monostearate, ethylene glycol distearate, glyceryl monostearate, and propylene glycol monostearate, polyvinyl alcohol), carbomers (e.g. carboxy polymethylene, polyacrylic acid, acrylic acid polymer, and carboxyvinyl polymer), carrageenan, cellulosic derivatives (e.g. carboxymethyl cellulose sodium, powdered cellulose, hydroxymethyl cellulose, hydroxypropyl cellulose, hydroxypropyl methylcellulose, methylcellulose), sorbitan fatty acid esters (e.g. polyoxyethylene sorbitan monolaurate (Tween 20), polyoxyethylene sorbitan (Tween 60), polyoxyethylene sorbitan monooleate (Tween 80), sorbitan monopalmitate (Span 40), sorbitan monostearate (Span 60), sorbitan tristearate (Span 65), glyceryl monooleate, sorbitan monooleate (Span 80)), polyoxyethylene esters (e.g. polyoxyethylene monostearate (Myrj 45), polyoxyethylene hydrogenated castor oil, polyethoxylated castor oil, polyoxymethylene stearate, and Solutol), sucrose fatty acid esters, polyethylene glycol fatty acid esters (e.g. CremophorTM) polyoxyethylene ethers, (e.g. polyoxyethylene lauryl ether (Brij 30)), poly(vinyl-pyrrolidone), diethylene glycol monolaurate, triethanolamine oleate, sodium oleate, potassium oleate, ethyl oleate, oleic acid, ethyl laurate, sodium lauryl sulfate, Pluronic F-68, Poloxamer-188, cetrimonium bromide, cetylpyridinium chloride, benzalkonium chloride, docusate sodium, and/or mixtures thereof.

Exemplary binding agents include starch (e.g. cornstarch and starch paste), gelatin, sugars (e.g. sucrose, glucose, dextrose, dextrin, molasses, lactose, lactitol, mannitol, etc.), natural and synthetic gums (e.g. acacia, sodium alginate, extract of Irish moss, panwar gum, ghatti gum, mucilage of isapol husks, carboxymethyl cellulose, methylcellulose, ethylcellulose, hydroxyethylcellulose, hydroxypropyl cellulose, hydroxypropyl methylcellulose, microcrystalline cellulose, cellulose acetate, poly(vinyl-pyrrolidone), magnesium aluminum silicate (Veegum), and larch arabogalactan), alginates, polyethylene oxide, polyethylene glycol, inorganic calcium salts, silicic acid, polymethacrylates, waxes, water, alcohol, and/or mixtures thereof.

Exemplary preservatives include antioxidants, chelating agents, antimicrobial preservatives, antifungal preservatives, alcohol preservatives, acidic preservatives, and other preservatives. In certain embodiments, the preservative is an antioxidant. In other embodiments, the preservative is a chelating agent.

Exemplary antioxidants include alpha tocopherol, ascorbic acid, acorbyl palmitate, butylated hydroxyanisole, butylated hydroxytoluene, monothioglycerol, potassium metabisulfite, propionic acid, propyl gallate, sodium ascorbate, sodium bisulfite, sodium metabisulfite, and sodium sulfite.

Exemplary chelating agents include ethylenediaminetetraacetic acid (EDTA) and salts and hydrates thereof (e.g., sodium edetate, disodium edetate, trisodium edetate, calcium disodium edetate, dipotassium edetate, and the like), citric acid and salts and hydrates thereof (e.g., citric acid monohydrate), fumaric acid and salts and hydrates thereof, malic acid and salts and hydrates thereof, phosphoric acid and salts and hydrates thereof, and tartaric acid and salts and hydrates thereof. Exemplary antimicrobial preservatives include benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and thimerosal.

Exemplary antifungal preservatives include butyl paraben, methyl paraben, ethyl paraben, propyl paraben, benzoic acid, hydroxybenzoic acid, potassium benzoate, potassium sorbate, sodium benzoate, sodium propionate, and sorbic acid.

Exemplary alcohol preservatives include ethanol, polyethylene glycol, phenol, phenolic compounds, bisphenol, chlorobutanol, hydroxybenzoate, and phenylethyl alcohol.

Exemplary acidic preservatives include vitamin A, vitamin C, vitamin E, beta-carotene, citric acid, acetic acid, dehydroacetic acid, ascorbic acid, sorbic acid, and phytic acid.

Other preservatives include tocopherol, tocopherol acetate, deteroxime mesylate, cetrimide, butylated hydroxyanisol (BHA), butylated hydroxytoluened (BHT), ethylenediamine, sodium lauryl sulfate (SLS), sodium lauryl ether sulfate (SLES), sodium bisulfite, sodium metabisulfite, potassium sulfite, potassium metabisulfite, Glydant Plus, Phenonip, methylparaben, Germall 115, Germaben II, Neolone, Kathon, and Euxyl.

Exemplary buffering agents include citrate buffer solutions, acetate buffer solutions, phosphate buffer solutions, ammonium chloride, calcium carbonate, calcium chloride, calcium citrate, calcium glubionate, calcium gluceptate, calcium gluconate, D-gluconic acid, calcium glycerophosphate, calcium lactate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, magnesium hydroxide, aluminum hydroxide, alginic acid, pyrogen-free water, isotonic saline, Ringer's solution, ethyl alcohol, and mixtures thereof.

Exemplary lubricating agents include magnesium stearate, calcium stearate, stearic acid, silica, talc, malt, glyceryl behanate, hydrogenated vegetable oils, polyethylene glycol, sodium benzoate, sodium acetate, sodium chloride, leucine, magnesium lauryl sulfate, sodium lauryl sulfate, and mixtures thereof.

Exemplary natural oils include almond, apricot kernel, avocado, babassu, bergamot, black current seed, borage, cade, camomile, canola, caraway, carnauba, castor, cinnamon, cocoa butter, coconut, cod liver, coffee, corn, cotton seed, emu, eucalyptus, evening primrose, fish, flaxseed, geraniol, gourd, grape seed, hazelnut, hyssop, isopropyl myristate, jojoba, kukui nut, lavandin, lavender, lemon, litsea cubeba, macademia nut, mallow, mango seed, meadowfoam seed, mink, nutmeg, olive, orange, orange roughy, palm, palm kernel, peach kernel, peanut, poppy seed, pumpkin seed, rapeseed, rice bran, rosemary, safflower, sandalwood, sasquana, savoury, sea buckthorn, sesame, shea butter, silicone, soybean, sunflower, tea tree, thistle, tsubaki, vetiver, walnut, and wheat germ oils. Exemplary synthetic oils include, but are not limited to, butyl stearate, caprylic triglyceride, capric triglyceride, cyclomethicone, diethyl sebacate, dimethicone 360, isopropyl myristate, mineral oil, octyldodecanol, oleyl alcohol, silicone oil, and mixtures thereof.

Liquid dosage forms for oral administration include, but are not limited to, pharmaceutically acceptable emulsions, microemulsions, solutions, suspensions, syrups, and elixirs. In addition to the active agents, the liquid dosage forms may contain inert diluents commonly used in the art such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, oral compositions can also include adjuvants such as wetting agents, emulsifying and suspending agents, sweetening, flavoring, and perfuming agents. In certain embodiments for parenteral administration, agents of the invention are mixed with solubilizing agents such CREMOPHOR EL® (polyethoxylated castor oil), alcohols, oils, modified oils, glycols, polysorbates, cyclodextrins, polymers, and combinations thereof.

Compositions for rectal or vaginal administration are typically suppositories which can be prepared by mixing the conjugates described herein with suitable non-irritating excipients or carriers such as cocoa butter, polyethylene glycol, or a suppository wax which are solid at ambient temperature but liquid at body temperature and therefore melt in the rectum or vaginal cavity and release the active ingredient.

Solid dosage forms for oral administration include capsules, tablets, pills, powders, and granules. In such solid dosage forms, the active agent is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethyl cellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions which can be used include polymeric substances and waxes. Solid compositions of a similar type may also be employed as fillers in soft and hard-filled gelatin capsules using such excipients as lactose or milk sugar as well as high molecular weight polyethylene glycols and the like.

The active agents can also be in micro-encapsulated form with one or more excipients as noted above. The solid dosage forms of tablets, dragees, capsules, pills, and granules can be prepared with coatings and shells such as enteric coatings, release controlling coatings and other coatings well known in the pharmaceutical formulating art. In such solid dosage forms the active agent may be admixed with at least one inert diluent such as sucrose, lactose or starch. Such dosage forms may also comprise, as is normal practice, additional substances other than inert diluents, e.g., tableting lubricants and other tableting aids such a magnesium stearate and microcrystalline cellulose. In the case of capsules, tablets, and pills, the dosage forms may also comprise buffering agents. They may optionally contain opacifying agents and can also be of a composition that they release the active ingredient(s) only, or preferentially, in a certain part of the intestinal tract, optionally, in a delayed manner. Examples of embedding compositions which can be used include polymeric substances and waxes.

Formulations suitable for topical administration include sprays, inhalants, liniments, lotions, gels, applicants, oil-in-water or water-in-oil emulsions such as creams, ointments, or pastes; or solutions or suspensions such as drops. Formulations for topical administration to the skin surface can be prepared by dispersing the drug with a dermatologically acceptable carrier such as a lotion, cream, ointment, or soap. Useful carriers are capable of forming a film or layer over the skin to localize application and inhibit removal. For topical administration to internal tissue surfaces, the agent can be dispersed in a liquid tissue adhesive or other substance known to enhance adsorption to a tissue surface. For example, hydroxypropylcellulose or fibrinogen/thrombin solutions can be used to advantage. Alternatively, tissue-coating solutions, such as pectin-containing formulations can be used. Ophthalmic formulation, ear drops, and eye drops are also contemplated as being within the scope of this invention. Additionally, the present disclosure contemplates the use of transdermal patches, which have the added advantage of providing controlled delivery of an agent to the body. Such dosage forms can be made by dissolving or dispensing the agent in the proper medium. Absorption enhancers can also be used to increase the flux of the agent across the skin. The rate can be controlled by either providing a rate controlling membrane or by dispersing the agent in a polymer matrix or gel. The present disclosure contemplates the use of washes (e.g., anti-infective washes) such as mouthwash or mouth rinse or application to surfaces in the oral cavity, such as the teeth. Dosage forms may also include toothpastes.

Additionally, the carrier for a topical formulation can be in the form of a hydroalcoholic system (e.g., quids and gels), an anhydrous oil or silicone based system, or an emulsion system, including, but not limited to, oil-in-water, water-in-oil, water-in-oil-in-water, and oil-in-water-in-silicone emulsions. The emulsions can cover a broad range of consistencies including thin lotions (which can also be suitable for spray or aerosol delivery), creamy lotions, light creams, heavy creams, and the like. The emulsions can also include microemulsion systems. Other suitable topical carriers include anhydrous solids and semisolids (such as gels and sticks); and aqueous based mousse systems.

After formulation with an appropriate pharmaceutically acceptable carrier in a desired dosage, the pharmaceutical compositions of the disclosure can be administered to humans and other animals via any suitable route, including oral, topical (as by powders, ointments, coatings, creams, and/or drops), bucal, transdermal, interdermal, rectal, intravaginal, intraperitoneal, mucosal, nasal, sublingual; by intratracheal instillation, bronchial instillation, and/or inhalation; and/or as an oral spray, nasal spray, and/or aerosolor the like, depending on the disease or condition being treated.

In certain embodiments, a pharmaceutical composition comprising a polymer of Formula (I) is administered, e.g., orally or topically, at dosage levels of each pharmaceutical composition sufficient to deliver from about 0.001 mg/kg to about 200 mg/kg in one or more dose administrations for one or several days (depending on the mode of administration). In certain embodiments, the effective amount per dose varies from about 0.001 mg/kg to about 200 mg/kg, about 0.001 mg/kg to about 100 mg/kg, about 0.01 mg/kg to about 100 mg/kg, from about 0.01 mg/kg to about 50 mg/kg, preferably from about 0.1 mg/kg to about 40 mg/kg, preferably from about 0.5 mg/kg to about 30 mg/kg, from about 0.01 mg/kg to about 10 mg/kg, from about 0.1 mg/kg to about 10 mg/kg, of subject body weight per day, one or more times a day, to obtain the desired therapeutic and/or prophylactic effect. In certain embodiments, the compounds described herein may be at dosage levels sufficient to deliver from about 0.001 mg/kg to about 200 mg/kg, from about 0.001 mg/kg to about 100 mg/kg, from about 0.01 mg/kg to about 100 mg/kg, from about 0.01 mg/kg to about 50 mg/kg, preferably from about 0.1 mg/kg to about 40 mg/kg, preferably from about 0.5 mg/kg to about 30 mg/kg, from about 0.01 mg/kg to about 10 mg/kg, from about 0.1 mg/kg to about 10 mg/kg, and more preferably from about 1 mg/kg to about 25 mg/kg, of subject body weight per day, one or more times a day, to obtain the desired therapeutic and/or prophylactic effect. The desired dosage may be delivered three times a day, two times a day, once a day, every other day, every third day, every week, every two weeks, every three weeks, or every four weeks. In certain embodiments, the desired dosage may be delivered using multiple administrations (e.g., two, three, four, five, six, seven, eight, nine, ten, eleven, twelve, thirteen, fourteen, or more administrations). In certain embodiments, the composition described herein is administered at a dose that is below the dose at which the agent causes non-specific effects.

In certain embodiments, the pharmaceutical composition is administered at a dose of about 0.001 mg to about 1000 mg per unit dose. In certain embodiments, the pharmaceutical composition is administered at a dose of about 0.01 mg to about 200 mg per unit dose. In certain embodiments, the pharmaceutical composition is administered at a dose of about 0.01 mg to about 100 mg per unit dose. In certain embodiments, pharmaceutical composition is administered at a dose of about 0.01 mg to about 50 mg per unit dose. In certain embodiments, the pharmaceutical composition is administered at a dose of about 0.01 mg to about 10 mg per unit dose. In certain embodiments, the pharmaceutical composition is administered at a dose of about 0.1 mg to about 10 mg per unit dose.

In certain embodiments, formulations for inhalation may, for example, comprise from about as little as 0.1% (w/w) to as much as 100% (w/w) of the active ingredient, and may comprise one or more of the additional ingredients described herein. A pharmaceutical composition described herein can be prepared, packaged, and/or sold in a formulation for buccal administration. Such formulations may, for example, be in the form of tablets and/or lozenges made using conventional methods, and may contain, for example, 0.1 to 20% (w/w) active ingredient, the balance comprising an orally dissolvable and/or degradable composition and, optionally, one or more of the additional ingredients described herein. Alternately, formulations for buccal administration may comprise a powder and/or an aerosolized and/or atomized solution and/or suspension comprising the active ingredient. Such powdered, aerosolized, and/or aerosolized formulations, when dispersed, may have an average particle and/or droplet size in the range from about 0.1 to about 200 nanometers, and may further comprise one or more of the additional ingredients described herein.

A polymer or composition, as described herein, can be administered in combination with one or more additional pharmaceutical agents (e.g., therapeutically and/or prophylactically active agents). The compounds or compositions can be administered in combination with additional pharmaceutical agents (e.g., antibiotics) that improve their activity (e.g., activity (e.g., potency and/or efficacy) in treating a disease in a subject in need thereof, in preventing a disease in a subject in need thereof, and/or in reducing the risk to develop a disease in a subject in need thereof), improve bioavailability, improve their ability to cross the blood-brain barrier, improve safety, reduce drug resistance, reduce and/or modify metabolism, inhibit excretion, and/or modify distribution in a subject or cell. It will also be appreciated that the therapy employed may achieve a desired effect for the same disorder, and/or it may achieve different effects. In certain embodiments, a pharmaceutical composition described herein including a polymer described herein and an additional pharmaceutical agent exhibit a synergistic effect that is absent in a pharmaceutical composition including one of the compound and the additional pharmaceutical agent, but not both.

The polymer or composition can be administered concurrently with, prior to, or subsequent to one or more additional pharmaceutical agents, which may be useful as, e.g., combination therapies. Pharmaceutical agents include therapeutically active agents. Pharmaceutical agents also include prophylactically active agents. Pharmaceutical agents include small organic molecules such as drug compounds (e.g., compounds approved for human or veterinary use by the U.S. Food and Drug Administration as provided in the Code of Federal Regulations (CFR)), peptides, proteins, carbohydrates, monosaccharides, oligosaccharides, polysaccharides, nucleoproteins, mucoproteins, lipoproteins, synthetic polypeptides or proteins, small molecules linked to proteins, glycoproteins, steroids, nucleic acids, DNAs, RNAs, nucleotides, nucleosides, oligonucleotides, antisense oligonucleotides, lipids, hormones, vitamins, and cells. In certain embodiments, the additional pharmaceutical agent is a pharmaceutical agent useful for treating and/or preventing an infection (e.g., bacterial, protozoan, and/or fungal). Each additional pharmaceutical agent may be administered at a dose and/or on a time schedule determined for that pharmaceutical agent. The additional pharmaceutical agents may also be administered together with each other and/or with the polymer or composition described herein in a single dose or administered separately in different doses. The particular combination to employ in a regimen will take into account compatibility of the polymer described herein with the additional pharmaceutical agent(s) and/or the desired therapeutic and/or prophylactic effect to be achieved. In general, it is expected that the additional pharmaceutical agent(s) in combination be utilized at levels that do not exceed the levels at which they are utilized individually. In some embodiments, the levels utilized in combination will be lower than those utilized individually.

The present disclosure also provides prebiotic compositions comprising a polymer of Formula (I), or a salt thereof, and optionally an acceptable carrier. In certain embodiments, the prebiotic composition described herein comprises a polymer of Formula (I), or a salt thereof, and a carrier.

Prebiotics refer to food components that selectively stimulate the growth and/or activity of one or a limited number of beneficial bacteria in the colon, resulting in an improvement or maintenance of host health. The term “gut flora” and “microflora” refer to microorganisms that normally live in the digestive tract of a human or other animal. The human gut flora comprises pathogenic, benign, and beneficial microbial genera. For example, the gut flora of a normal, healthy animal may comprise beneficial bacteria such as lactobacilli, bifidobacteria, and non beneficial gut bacteria include bacteroides, coliforms, clostridia, and sulfate-reducing bacteria. A predominance of the latter can lead to intestinal disorders, acute or chronic, including gastroenteritis, inflammatory bowel syndrome, irritable bowel syndrome, and some intestinal cancers.

The prebiotic compositions may beneficially affect the host by selectively stimulating the growth and/or the activity of one or more of the beneficial bacteria in the colon, thereby resulting in an improvement in the health of the host. For example, a mammal being administered a prebiotic composition may experience the benefits of maintaining gastrointestinal health, improving gastrointestinal health, reducing cholesterol, attenuating blood dextrose, improving mineral absorption, or combinations thereof.

In certain embodiments, an effective amount of the prebiotic composition may be administered to a subject and function as a prebiotic to confer beneficial health effects. Such beneficial health effects may include digestive resistance, lower gut fermentation, selective promotion of beneficial microflora (e.g., lactobacilli and/or bifidobacteria) and reduction of pathogenic or nonbeneficial microflora (e.g., bacteroides, coliforms, clostridia, and/or sulfate-reducing bacteria). Without wishing to be limited by theory, prebiotic compositions of the type described herein may confer beneficial health effects by any number of mechanisms, nonlimiting examples of which include competitive exclusion and/or pathogen binding and/or site colonization interference, production of short chain fatty acids and/or decrease in pH in the gastrointestinal (GI) tract of the subject to which it is introduced.

In certain embodiments, a prebiotic composition may be administered to a subject in order to confer beneficial health effects of the type described herein. Alternatively, the prebiotic composition may be administered to a subject experiencing or anticipated to experience one or more adverse health events for which a prebiotic would ameliorate, mitigate, or prevent said adverse health event. For example, a prebiotic composition may be administered to a subject experiencing an adverse health event involving alterations in the gut flora. Alternatively, a prebiotic composition may be administered to a subject having an expectation of developing an adverse health event involving alterations in the gut flora. For example, a subject having been administered a pharmaceutical composition (e.g., an antibiotic) may have an increased probability of developing one or more symptoms of gastrointestinal distress (e.g., diarrhea) associated with the use of the antibiotic. In an embodiment, an effective amount of a prebiotic composition may be coadminstered with the pharmaceutical composition. Alternatively, an effective amount of a prebiotic composition may be administered prior to and/or subsequent to administration of the pharmaceutical composition. In either embodiment, the administration of a prebiotic composition may mitigate or prevent the development of gastrointestinal distress associated with the use of the pharmaceutical composition.

The term “effective amount” as used herein means that amount of the prebiotic composition that elicits the biological or medicinal response in a tissue, system, animal or human that is being sought by a researcher, veterinarian, medical doctor, or other clinician. In one embodiment, the effective amount is a “therapeutically effective amount” for the alleviation of the symptoms of the disease or condition being treated. In another embodiment, the effective amount is a “prophylactically effective amount” for prophylaxis of the symptoms of the disease or condition being prevented. It is contemplated that the compositions of the present disclosure may also be introduced to a subject in amounts less than a predetermined therapeutically and/or prophylatically effective amount. For example, a sub-effective amount of the disclosed compositions may be administered as an admixture of the composition with one or more food products and may serve to alter various properties of the food product (e.g., texture, appearance, taste, etc.).

In certain embodiments, the present disclosure provides a nutraceutical composition comprising a polymer of Formula (I), or a salt thereof, and optionally an acceptable carrier. In certain embodiments, the nutraceutical composition described herein comprises a polymer of Formula (I), or a salt thereof, and a carrier.

Nutraceuticals are products derived from food sources that provide health benefits, in addition to the basic nutritional value found in foods. Nutraceuticals may be a dietary supplement. A dietary supplement is a product that contains nutrients derived from food products that are concentrated in liquid or capsule form. A dietary supplement is a product taken by mouth that contains a dietary ingredient intended to supplement the diet. The dietary ingredients in these products may include: vitamins, minerals, herbs or other botanicals, amino acids, and substances such as enzymes, organ tissues, glandulars, and metabolites. Dietary supplements can also be extracts or concentrates, and may be found in many forms such as tablets, capsules, softgels, gelcaps, liquids, or powders. Nutraceuticals may also be a functional food. Functional foods are fortified or enriched during processing and then marketed as providing some benefit to consumers. Sometimes, additional complementary nutrients are added, such as vitamin D to milk. Functional foods are ordinary food that has components or ingredients added to give it a specific medical or physiological benefit, other than a purely nutritional effect. Functional foods may comprise foods and regulate a biological process for preventing or controlling disease.

The present disclosure also provides food products comprising a polymer of Formula (I), or a salt thereof. In certain embodiments, the food product comprises any of the compositions described herein (e.g., pharmaceutical, prebiotic, nutraceutical).

Any suitable route of administration may be employed for providing a subject (e.g., human or animal) a prebiotic composition, a nutraceutical composition, and/or a food product. Dosage forms include tablets, troches, dispersions, suspensions, solutions, capsules, solid foods, liquid foods, beverages, and the like. The most suitable route in any given case will depend on the nature and severity of the conditions being treated and on the nature of the active ingredient. They may be conveniently presented in unit dosage form and prepared by any of the methods well-known in the art of pharmacy and food preparation.

In practical use, a prebiotic or nutraceutical composition can be combined as the active ingredient in intimate admixture with a carrier according to conventional compounding techniques. The carrier may take a wide variety of forms depending on the form of preparation desired for administration. In preparing the compositions for oral dosage form, any of the usual pharmaceutical media may be employed, such as, for example, water, glycols, oils, alcohols, flavoring agents, preservatives, coloring agents and the like in the case of oral liquid preparations, such as, for example, suspensions, elixirs and solutions; or carriers such as starches, sugars, microcrystalline cellulose, diluents, granulating agents, lubricants, binders, disintegrating agents and the like in the case of oral solid preparations such as, for example, powders, capsules and tablets. Because of their ease of administration, tablets and capsules represent the most advantageous oral dosage unit form in which case solid pharmaceutical carriers may be employed. If desired, tablets may be coated by standard aqueous or nonaqueous techniques.

In certain embodiments, a suitable orally ingestible form comprises a composition (e.g., pharmaceutical, prebiotic, or nutraceutical) incorporated within a food, feed, or fodder product. The composition (e.g., pharmaceutical, prebiotic, or nutraceutical) may be incorporated within the food, feed, or fodder product as a dry powder or a liquid. Non-limiting examples of food, feed, or fodder products into which the composition (e.g., pharmaceutical, prebiotic, or nutraceutical) may be incorporated include compound feeds and premixes, such as pellets, nuts, nuggets, oil cakes, press cakes, various meals (e.g., fishmeal), or combinations thereof. Such food, feed, or fodder product may be prepared by admixing or blending the composition (e.g., pharmaceutical, prebiotic, or nutraceutical) with a suitable carrier or diluent. Non-limiting examples of suitable carriers may include grass and other forage plants, plant oils, seeds, grains, crop residues, sprouted grains, legumes, alfalfa meal, soybean meal, cottonseed oil meal, linseed oil meal, sodium chloride, cornmeal, molasses, urea, corncob meal, rice kernel, and the like. The carrier promotes a uniform distribution of the active ingredients in the finished feed into which the carrier is blended. It thus may ensure proper distribution of the active ingredient throughout the food, feed, or fodder product.

In certain embodiments, a suitable orally ingestible form comprises a composition (e.g., pharmaceutical, prebiotic, or nutraceutical) prepared as a nutritional supplement. Such a nutritional supplement may be ingestible by a subject alone or with another food, feed, fodder, forage product, snack, treat, or enjoyment product. In various embodiments, nutritional supplements may be prepared in a wet, semi-wet, or dry form. Nonlimiting examples of suitable nutritional supplement forms include powders, granules, syrups, and pills; other suitable forms will be known to those of skill in the art with the aid of this disclosure. In an embodiment, a nutritional supplement may be added to another food, feed, fodder, or forage product. For example, the nutritional supplement may comprise a powder or syrup which is dispensed with (e.g., poured onto) hay, pellets, forage, or the like. Alternatively, in an embodiment a nutritional supplement is provided without any other food or nutrient. For example, the nutritional supplement may comprise a syrup or gel which may be licked by a subject (e.g., from a tub or other suitable dispenser) or water-soluble powder dissolved in water provided for ingestion by the organism. Other suitable means of dispensing a nutritional supplement will be appreciated by those of skill in the art.

As will be appreciated by those of skill in the art, the ingestible forms may be formulated for ingestion by one or more subjects, nonlimiting examples of which include humans, livestock such as cattle, swine, horses, sheep, goats, poultry, fish, domesticated companionship species such as dogs, cats, fish, and rodents or undomesticated wildlife such as deer, moose, elk, migratory and non-migratory fowl, decapods, and fish.

In certain embodiments, administration of a composition (e.g., pharmaceutical, prebiotic, or nutraceutical) improves the overall health of the organism to which it is administered. In some embodiments, the overall improved health of the organism may be evidenced by an increase in biological functions such as nutrient uptake, muscle growth, muscle development, weight gain, coat growth, survival, or combinations thereof. In another embodiment, administration of the prebiotic composition to a subject results in an increased yield in a subject derived commodity such as eggs, meat, milk, wool, or combinations thereof.

### Kits

Also provided by the present disclosure are kits (e.g., pharmaceutical packs, food packages, nutraceutical packs). The kits provided may comprise a composition (e.g., pharmaceutical, prebiotic, nutraceutical, food, food supplement) or polymer described herein and a container (e.g., a vial, ampule, bottle, syringe, and/or dispenser package, or other suitable container). In some embodiments, provided kits may optionally further include a second container comprising a excipient for dilution or suspension of a composition or polymer described herein. In some embodiments, the composition or polymer described herein provided in the first container and the second container are combined to form one unit dosage form.

Thus, in one aspect, provided are kits including a first container comprising a polymer or composition (e.g., pharmaceutical, prebiotic, nutraceutical, food, food supplement) described herein. In certain embodiments, the kits are useful for treating an infection (e.g., bacterial, protozoan, fungal) in a subject in need thereof. In certain embodiments, the kits are useful for preventing an infection (e.g., bacterial, protozoan, fungal) in a subject in need thereof. In certain embodiments, the kits are useful for reducing the risk of developing an infection (e.g., bacterial, protozoan, fungal) in a subject in need thereof. In certain embodiments, the kits are useful for inhibiting the virulence of a microbe (e.g., bacteria, archaea, protozoa, fungi, algae). In certain embodiments, the kits are useful for inhibiting or preventing the formation of a biofilm on a surface. In certain embodiments, the kits are useful for inhibiting biofilm formation on a surface. In certain embodiments, the kits are useful for preventing biofilm formation on a surface. In certain embodiments, the kits are useful for improving gastrointestinal health of a subject.

In certain embodiments, a kit described herein further includes instructions for using the kit. A kit described herein may also include information as required by a regulatory agency such as the U.S. Food and Drug Administration (FDA). In certain embodiments, the information included in the kits is prescribing information. In certain embodiments, the kits and instructions provide for treating an infection (e.g., bacterial, protozoan, fungal) in a subject in need thereof. In certain embodiments, the kits and instructions provide for preventing an infection (e.g., bacterial, protozoan, fungal) in a subject in need thereof. In certain embodiments, the kits and instructions provide for reducing the risk of developing an infection (e.g., bacterial, protozoan, fungal) in a subject in need thereof. In certain embodiments, the kits and instructions provide for inhibiting the virulence of a microbe (e.g., bacteria, archaea, protozoa, fungi, algae). In certain embodiments, the kits and instructions provide for inhibiting or preventing biofilm formation on a surface. In certain embodiments, the kits and instructions provide for inhibiting biofilm formation on a surface. In certain embodiments, the kits and instructions provide for preventing biofilm formation on a surface. In certain embodiments, the kits and instructions provide for improving gastrointestinal health of a subject. A kit described herein may include one or more additional pharmaceutical agents described herein as a separate composition.

### Methods of Treatment

Biofilms have been estimated to be involved in up to 80% of all microbial infections in the body. Infectious processes in which biofilms have been implicated include conditions such as bacterial vaginosis, urinary tract infections, catheter infections, middle-ear infections, formation of dental plaque, gingivitis, coating contact lenses, endocarditis, infections in cystic fibrosis, and infections of permanent indwelling devices such as joint prostheses, heart valves, and intervertebral disc. Bacterial biofilms may impair cutaneous wound healing and reduce topical antibacterial efficiency in healing or treating infected skin wounds. Early detection of biofilms in wounds is important to successful chronic wound management. Although many techniques have been developed to identify planktonic bacteria in viable wounds, few have been able to quickly and accurately identify bacterial biofilms.

The human oral microbial biota represents a highly diverse biofilm. Twenty-five species of oral streptococci inhabit the human oral cavity, represent about 20% of the total oral bacteria, and determine the development of biofilms. Each species has developed specific properties for colonizing the different oral sites subjected to constantly changing conditions, for competing against competitors, and for resisting external agressions (host immune system, physico-chemical shocks, and mechanical frictions). Imbalance in the indigenous microbial biota generates oral diseases, and under proper conditions, commensal Streptococci can switch to opportunistic pathogens that initiate disease in and damage to the host. The group of “mutans streptococci” was described as the most important bacteria related to the formation of dental caries. Streptococcus mutans, although naturally present among the human oral microbiota, is the microbial species most strongly associated with carious lesions.

S. mutans has, over time, developed strategies to successfully colonize and maintain a dominant presence in the oral cavity. The oral biofilm is continuously challenged by changes in the environmental conditions. In response to such changes, the bacterial community evolved with individual members and their specific functions to survive in the oral cavity. S. mutans has evolved from nutrition-limiting conditions to protect itself in extreme conditions. Although S. mutans can be antagonized by pioneer colonizers, once they become dominant in oral biofilms, dental caries can develop and thrive.

Dental caries are associated with increased consumption of dietary sugar and fermentable carbohydrates. When dental biofilms remain on tooth surfaces, along with frequent exposure to sugars, acidogenic bacteria (members of dental biofilms) will metabolize the sugars to organic acids. Persistence of this acidic condition encourages the proliferation of acidogenic and aciduric bacteria as a result of their ability to survive at a low-pH environment. The low-pH environment in the biofilm matrix erodes the surface of the teeth and begins the initiation of the dental caries. If the adherence of S. mutans to the surface of teeth or the physiological ability (acidogenity and aciduricity) of S. mutans in dental biofilms can be reduced or eliminated, the acidification potential of dental biofilms and later cavity formations can be decreased.

In addition, the rapidly expanding industry for biomedical devices and tissue engineering related products continues to suffer from microbial colonization. Despite increased sophistication, microbial infections can develop on all medical devices and tissue engineering constructs (e.g., inert surfaces of implanted devices such as catheters, prosthetic cardiac valves and intrauterine devices), and 60-70% of hospital acquired infections are associated with the implantation of a biomedical device. If an infection develops a biofilm, it becomes even more difficult to treat. As the bacteria change, they becomes more resistant to antibiotics and the body's own host defenses.

Accordingly, use of polymers that inhibit and/or prevent formation of biofilms that include pathogenic microbes provides a strategy for treating infections and/or diseases associated with these biofilms.

The present disclosure provides methods for inhibiting the virulence of a microbe. In certain embodiments, the microbe is bacteria, archaea, protozoa, fungi, or algae.

The present disclosure provides methods for inhibiting or preventing biofilm formation on a surface. In certain embodiments, the disclosure provides methods for inhibiting biofilm formation on a surface. In certain embodiments, the disclosure provides methods for preventing biofilm formation on a surface. In certain embodiments, the biofilm comprises bacteria, archaea, protozoa, fungi, or algae. In certain embodiments, the biofilm is caused by bacteria, archaea, protozoa, fungi, or algae. In certain embodiments, the biofilm comprises Entamoeba histolytica, Plasmodium, Giardia lamblia, Trypanosoma brucei, Aspergillus, Blastomyces, Candida (e.g., Candida albicans), Coccidioides, Cryptococcus (e.g., Cryptococcus gattii, Cryptococcus neoformans), Histoplasma, Pneumocystis jirovecii, Sporothrix, Exserohilum, Cladosporium, Actinomyces, Bacillus, Becteroides, Bordatella, Borrelia, Brucella, Camylobacter, Chlamydia, Chlamydophila, Clostridium, Corynebacterium, Ehrlichia, Enterococcus, Escherichia, Francisella, Haemophilus, Helicobacter, Klebsiella, Legionella, Leptospira, Listeria, Mycobacterium, Mycoplasma, Neisseria, Pseudomonas (e.g., Pseudomonas aeruginosa), Nocardia, Rickettsia, Salmonella, Shigella, Staphylococcus (e.g., Staphylococcus aureus), Streptococcus (e.g., Streptococcus mutans, Streptococcus pneumonia), Treponema, Vibrio, and/or Yersinia. In certain embodiments, the biofilm is caused by Entamoeba histolytica, Plasmodium, Giardia lamblia, Trypanosoma brucei, Aspergillus, Blastomyces, Candida (e.g., Candida albicans), Coccidioides, Cryptococcus (e.g., Cryptococcus gattii, Cryptococcus neoformans), Histoplasma, Pneumocystis jirovecii, Sporothrix, Exserohilum, Cladosporium, Actinomyces, Bacillus, Becteroides, Bordatella, Borrelia, Brucella, Camylobacter, Chlamydia, Chlamydophila, Clostridium, Corynebacterium, Ehrlichia, Enterococcus, Escherichia, Francisella, Haemophilus, Helicobacter, Klebsiella, Legionella, Leptospira, Listeria, Mycobacterium, Mycoplasma, Neisseria, Pseudomonas (e.g., Pseudomonas aeruginosa), Nocardia, Rickettsia, Salmonella, Shigella, Staphylococcus (e.g., Staphylococcus aureus), Streptococcus (e.g., Streptococcus mutans, Streptococcus pneumonia), Treponema, Vibrio, and/or Yersinia. In certain embodiments, the biofilm comprises Streptococcus mutans, Pseudomonas aeruginosa, Staphylococcus aureus, or Candida albicans. In certain embodiments, the biofilm is caused by Streptococcus mutans, Pseudomonas aeruginosa, Staphylococcus aureus, or Candida albicans. In certain embodiments, the biofilm comprises Streptococcus mutans. In certain embodiments, the biofilm is caused by Streptococcus mutans.

In certain embodiments, the surface being treated is a surface in or on a subject (e.g., tooth, wound). In certain embodiments, the surface being treated is a surface on an inanimate object (e.g., medical device). In certain embodiments, medical devices can be coated with a polymer or composition of the present disclosure to inhibit the virulence of microbes on the surface and reduce or prevent any infection described herein. In certain embodiments, surfaces in or on a subject are coated with a polymer or composition of the present disclosure. In certain embodiments, teeth of a subject are coated with a polymer or composition of the present disclosure to reduce or prevent plaque formation.

The present disclosure provides methods for treating infections. The present disclosure provides methods for preventing infections. In certain embodiments, the infection is a bacterial, protozoan, or fungal infection. In certain embodiments, the infection is a protozoan infection. In certain embodiments, the protozoan infection is caused by Entamoeba histolytica, Plasmodium, Giardia lamblia, and/or Trypanosoma brucei. In certain embodiments, the infection is a fungal infection. In certain embodiments, the fungal infection is caused by Aspergillus, Blastomyces, Candida (e.g., Candida albicans), Coccidioides, Cryptococcus (e.g., Cryptococcus gattii, Cryptococcus neoformans), Histoplasma, Pneumocystis jirovecii, Sporothrix, Exserohilum and/or Cladosporium. In certain embodiments, the infection is a bacterial infection. In certain embodiments, the bacterial infection is caused by Actinomyces, Bacillus, Becteroides, Bordatella, Borrelia, Brucella, Camylobacter, Chlamydia, Chlamydophila, Clostridium, Corynebacterium, Ehrlichia, Enterococcus, Escherichia, Francisella, Haemophilus, Helicobacter, Klebsiella, Legionella, Leptospira, Listeria, Mycobacterium, Mycoplasma, Neisseria, Pseudomonas (e.g., Pseudomonas aeruginosa), Nocardia, Rickettsia, Salmonella, Shigella, Staphylococcus (e.g., Staphylococcus aureus), Streptococcus (e.g., Streptococcus mutans, Streptococcus pneumonia), Treponema, Vibrio, and/or Yersinia. In certain embodiments, the infection is caused by Streptococcus mutans, Pseudomonas aeruginosa, Staphylococcus aureus, or Candida albicans. In certain embodiments, the infection is caused by Streptococcus mutans.

The present disclosure also provides methods for improving gastrointestinal health.

In certain embodiments, the methods comprise administering a polymer of Formula (I), or a salt, or composition thereof, to a subject in need thereof. In some embodiments, the method comprises administering a pharmaceutical composition comprising a polymer of Formula (I), or a salt, or composition thereof, to a subject in need thereof. In some embodiments, the method comprises administering a prebiotic composition comprising a polymer of Formula (I), or a salt, or composition thereof, to a subject in need thereof. In some embodiments, the method comprises administering a nutraceutical composition comprising a polymer of Formula (I), or a salt, or composition thereof, to a subject in need thereof. In some embodiments, the method comprises administering a food product comprising a polymer of Formula (I), or a salt, or composition thereof, to a subject in need thereof.

The present disclosure also provides a polymer of Formula (I), or a salt thereof, for use in inhibiting the virulence of a microbe. In certain embodiments, the microbe is bacteria, archaea, protozoa, fungi or algae.

The present disclosure also provides a polymer of Formula (I), or a salt thereof, for use in inhibiting or preventing biofilm formation on a surface. The present disclosure also provides a polymer of Formula (I), or a salt thereof, for use in inhibiting biofilm formation on a surface. The present disclosure also provides a polymer of Formula (I), or a salt thereof, for use in preventing biofilm formation on a surface. In certain embodiments, the biofilm comprises bacteria, archaea, protozoa, fungi, or algae. In certain embodiments, the biofilm is caused by bacteria, archaea, protozoa, fungi, or algae. In certain embodiments, the biofilm comprises Streptococcus mutans, Pseudomonas aeruginosa, Staphylococcus aureus, or Candida albicans. In certain embodiments, the biofilm is caused by Streptococcus mutans, Pseudomonas aeruginosa, Staphylococcus aureus, or Candida albicans. In certain embodiments, the biofilm comprises Streptococcus mutans. In certain embodiments, the biofilm is caused by Streptococcus mutans.

The present disclosure also provides a polymer of Formula (I), or a salt thereof, for use in treating infections. The present disclosure also provides a polymer of Formula (I), or a salt thereof, for use in preventing infections. In certain embodiments, the infection is a bacterial, protozoan, or fungal infection. In certain embodiments, the infection is a protozoan infection. In certain embodiments, the protozoan infection is caused by Entamoeba histolytica, Plasmodium, Giardia lamblia, and/or Trypanosoma brucei. In certain embodiments, the infection is a fungal infection. In certain embodiments, the fungal infection is caused by Aspergillus, Blastomyces, Candida (e.g., Candida albicans), Coccidioides, Cryptococcus (e.g., Cryptococcus gattii, Cryptococcus neoformans), Histoplasma, Pneumocystis jirovecii, Sporothrix, Exserohilum, and/or Cladosporium. In certain embodiments, the infection is a bacterial infection. In certain embodiments, the bacterial infection is caused by Actinomyces, Bacillus, Becteroides, Bordatella, Borrelia, Brucella, Camylobacter, Chlamydia, Chlamydophila, Clostridium, Corynebacterium, Ehrlichia, Enterococcus, Escherichia, Francisella, Haemophilus, Helicobacter, Klebsiella, Legionella, Leptospira, Listeria, Mycobacterium, Mycoplasma, Neisseria, Pseudomonas (e.g., Pseudomonas aeruginosa), Nocardia, Rickettsia, Salmonella, Shigella, Staphylococcus (e.g., Staphylococcus aureus), Streptococcus (e.g., Streptococcus mutans, Streptococcus pneumonia), Treponema, Vibrio, and/or Yersinia. In certain embodiments, the infection is caused by Streptococcus mutans, Pseudomonas aeruginosa, Staphylococcus aureus, or Candida albicans. In certain embodiments, the infection is caused by Streptococcus mutans.

The present disclosure also provides a polymer of Formula (I), or a salt thereof, for use in improving gastrointestinal health.

The present disclosure also provides uses of a polymer of Formula (I), or a salt thereof, in the manufacture of a medicament for inhibiting or preventing the formation of a biofilm on a surface. The present disclosure also provides uses of a polymer of Formula (I), or a salt thereof, in the manufacture of a medicament for inhibiting biofilm formation on a surface. The present disclosure also provides uses of a polymer of Formula (I), or a salt thereof, in the manufacture of a medicament for preventing biofilm formation on a surface. In certain embodiments, the biofilm comprises bacteria, archaea, protozoa, fungi, or algae. In certain embodiments, the biofilm is caused by bacteria, archaea, protozoa, fungi, or algae. In certain embodiments, the biofilm comprises Streptococcus mutans, Pseudomonas aeruginosa, Staphylococcus aureus, and/or Candida albicans. In certain embodiments, the biofilm is caused by Streptococcus mutans, Pseudomonas aeruginosa, Staphylococcus aureus, and/or Candida albicans. In certain embodiments, the biofilm comprises Streptococcus mutans. In certain embodiments, the biofilm is caused by Streptococcus mutans.

The present disclosure also provides uses of a polymer of Formula (I), or a salt thereof, in the manufacture of a medicament for the treatment of an infection. In certain embodiments, the infection is a bacterial, protozoan, or fungal infection. In certain embodiments, the infection is a protozoan infection. In certain embodiments, the protozoan infection is caused by Entamoeba histolytica, Plasmodium, Giardia lamblia, and/or Trypanosoma brucei. In certain embodiments, the infection is a fungal infection. In certain embodiments, the fungal infection is caused by Aspergillus, Blastomyces, Candida (e.g., Candida albicans), Coccidioides, Cryptococcus (e.g., Cryptococcus gattii, Cryptococcus neoformans), Histoplasma, Pneumocystis jirovecii, Sporothrix, Exserohilum and/or Cladosporium. In certain embodiments, the infection is a bacterial infection. In certain embodiments, the bacterial infection is caused by Actinomyces, Bacillus, Becteroides, Bordatella, Borrelia, Brucella, Camylobacter, Chlamydia, Chlamydophila, Clostridium, Corynebacterium, Ehrlichia, Enterococcus, Escherichia, Francisella, Haemophilus, Helicobacter, Klebsiella, Legionella, Leptospira, Listeria, Mycobacterium, Mycoplasma, Neisseria, Pseudomonas (e.g., Pseudomonas aeruginosa), Nocardia, Rickettsia, Salmonella, Shigella, Staphylococcus (e.g., Staphylococcus aureus), Streptococcus (e.g., Streptococcus mutans), Treponema, Vibrio, and/or Yersinia. In certain embodiments, the infection is caused by Streptococcus mutans, Pseudomonas aeruginosa, Staphylococcus aureus, or Candida albicans. In certain embodiments, the infection is caused by Streptococcus mutans.

In certain embodiments, the methods of the disclosure comprise administering to the subject an effective amount of a polymer of Formula (I), or a salt thereof. In some embodiments, the effective amount is a therapeutically effective amount. In some embodiments, the effective amount is a prophylactically effective amount.

In certain embodiments, the subject being treated is an animal. The animal may be of either sex and may be at any stage of development. In certain embodiments, the subject is a mammal. In certain embodiments, the subject being treated is a human. In certain embodiments, the subject is a domesticated animal, such as a dog, cat, cow, pig, horse, sheep, or goat. In certain embodiments, the subject is a companion animal, such as a dog or cat. In certain embodiments, the subject is a livestock animal, such as a cow, pig, horse, sheep, or goat. In certain embodiments, the subject is a zoo animal. In another embodiment, the subject is a research animal such as a rodent (e.g., mouse, rat), dog, pig, or non-human primate. In certain embodiments, the animal is a genetically engineered animal. In certain embodiments, the animal is a transgenic animal.

Certain methods described herein may comprise administering one or more additional pharmaceutical agent(s) in combination with the compounds described herein. The additional pharmaceutical agent(s) may be administered at the same time as the polymer of Formula (I), or at different times than the polymer of Formula (I). For example, the polymer of Formula (I) and any additional pharmaceutical agent(s) may be on the same dosing schedule or different dosing schedules. All or some doses of the polymer of Formula (I) may be administered before all or some doses of an additional pharmaceutical agent, after all or some does an additional pharmaceutical agent, within a dosing schedule of an additional pharmaceutical agent, or a combination thereof. The timing of administration of the polymer of Formula (I) and additional pharmaceutical agents may be different for different additional pharmaceutical agents.

In certain embodiments, the additional pharmaceutical agent comprises an agent useful in the treatment of an infection. In certain embodiments, the additional pharmaceutical agent is useful in the treatment of a bacterial, protozoan, or fungal infection. In certain embodiments, the additional pharmaceutical agent is an antibiotic. In certain embodiments, the additional pharmaceutical agent is useful in inhibiting and/or preventing biofilm formation on a surface.

### Other Uses of the Polymers

The polymers described herein may be useful in a variety of other applications. For example, the polymers may provide useful materials such as teeth whiteners, smell modifiers, lubricants, anti-fouling agents, and selective filters.

In certain embodiments, the present disclosure provides a lubricant comprising the polymer of Formula (I). The lubricant may be useful when deposited on epithelial surfaces, e.g., for the treatment of dry eye syndrome or dry mouth.

In other embodiments, the polymers may be useful for filtration and/or chromatography systems. In particular, polymers of Formula (I) may be used to develop novel chromatography systems for purification of compounds that specifically target or pass through mucosal tissues.

Additional uses will be self evident to one of ordinary skill in the art.

### EXAMPLES

These and other aspects of the present invention will be further appreciated upon consideration of the following Examples, which are intended to illustrate certain particular embodiments of the invention but are not intended to limit its scope, as defined by the claims.

### Materials and Methods

Unless otherwise stated, all reagents were obtained from Sigma Aldrich. The bacterial strain Streptococcus mutans UA159 was provided by Dan Smith (Forsyth Institute). For general propagation and storage, UA159 was cultured and maintained in Todd-Hewitt (TH, BD 249240) at 37° C. with 5% CO2. Plasmid DNA pVA838 (ATCC 37160) for transformation assays was purchased from ATCC and maintained in E. coli DH5a (BD LB medium, 10 ug/ml chloramphenicol). All TH media was used within seven days of autoclaving.

Synthesis of glycosylamines: The aldehyde group of reducing sugars was converted to a primary amine via the standard Kochetkov reaction. The amination was achieved by incubating the sugars with ammonia bicarbonate at a 1:40 w/w ratio for four days at room temperature (Kochetkov amination). The liquid containing the solubilized sugars was aspirated and collected. Excess ammonia was removed via repeated lyophilization until the weight stabilized. The glycosylamines were used fresh for conjugation to carboxymethyl cellulose (CMC) backbones.

Glycosylamine conjugation to CMC: Prior to conjugation, 90 kDa or 250 kDa CMC was dissolved overnight at 10 mg/mL concentrations. The effective molar concentrations of carboxyl groups were estimated based on the reported degrees of substitution (D.S.=1.2). Hydroxybenzotriazole (HOBt) and 1-ethyl-3-(3-dimethylaminopropyl)carbodiimide (EDC) were added at a 1:1:1 molar ratio to the carboxyl groups of the CMC and solubilized for up to 30 minutes with stirring. Once fully dissolved, glycosylamine was added at a 2:1 molar ratio to the carboxyl groups and stirred for 30 minutes at room temperature. Once the reaction was complete, the solution was diluted 10-fold with milliQ water to quench the reaction. Residual glycosylamine and reactants were removed by dialyzation using a 10 kDa cutoff membrane. Dialyzed glycosylated CMC was lyophilized and stored at room temperature in a desiccation chamber.

Horizontal Gene Transfer Assay: For all gene transfer assays, unless specified separately, UA159 cultures were grown overnight in 50% TH media at 37° C., 5% CO2 under static conditions. The next morning, cultures were diluted 1:20 into the 25% TH media in 100 uL volumes in 96-well plates and cultured statically for 2 hours. pVA838 (1.2 μg/ml unless otherwise noted) was added and gently mixed into the solution and allowed to incubate for an additional two hours before serially diluting 1:10 in phosphate buffer saline (PBS). For transformants, dilutions were plated on selective TH agar plates (10 μg/ml erythromycin). For total population counts, serial dilutions were plated on non-selective TH agar plates. All data presented are biological replicates with standard deviations reported for error bars.

Biofilm inhibition assay: For all biofilm inhibition assays, unless specified separately, UA159 cultures were grown overnight in 50% TH media at 37° C., 5% CO₂ under static conditions. The next morning, cultures were diluted 1:20 into the 25% TH media containing 1% (w/v) sterile sucrose in 100 μL volumes in 96-well plates and cultured statically for 4 hours. During the serial dilution process, the planktonic phase was gently pipetted and separated. The attached biofilm was washed three times by 100 μl of sterile PBS. Biofilms were then detached by vigorous scraping with a pipette tip for 30 seconds and mixing prior to serial dilution.

### Results

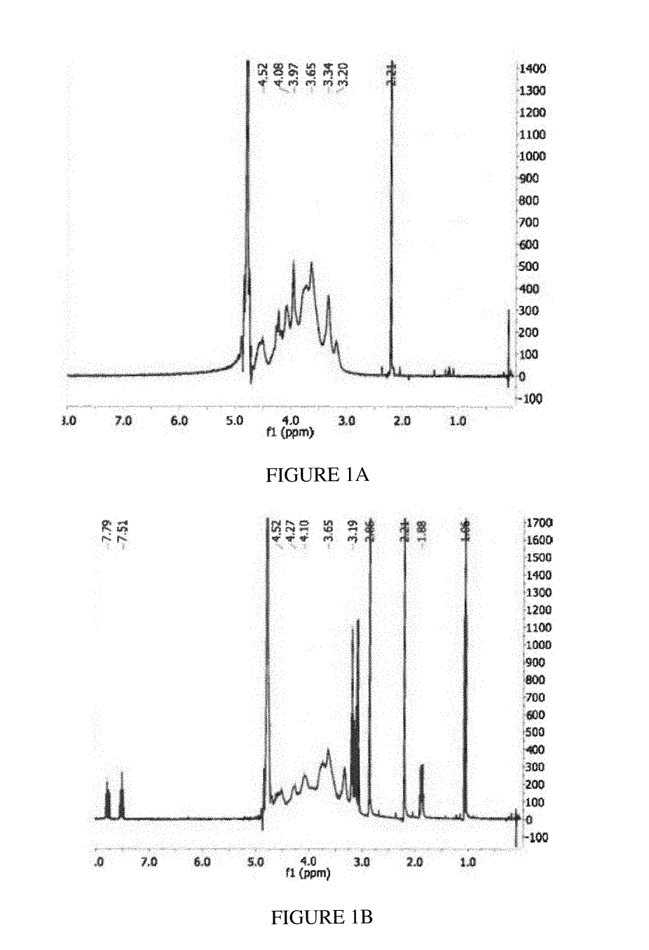

Several glycopolymers were prepared by glycosylation of 90 kDa CMC with glucose, galactose, mannose, and fucose (Glc-CMC, Gal-CMC, Man-CMC, and Fuc-CMC). Glycosylation of CMC with glucose was confirmed by ¹H NMR, as a diagnostic peak at 3.19 ppm was observed for Glc-CMC (FIGS. 1 and 1A). Using glucose as the proxy for all testing purposes, glucose oxidation assays were used to detect the extent of labeling. With this absorbance assay, the concentration of labeled glucose was determined to be about 0.1 mg per milligram of Glc-CMC, which is 10% of the molecular weight. These results reveal that not all possible carboxyl groups were glycosylated with the monosaccharide. Assuming that all the glycoslation densities were consistent for each of the tested sugars, the functionality of the glycopolymers were tested.

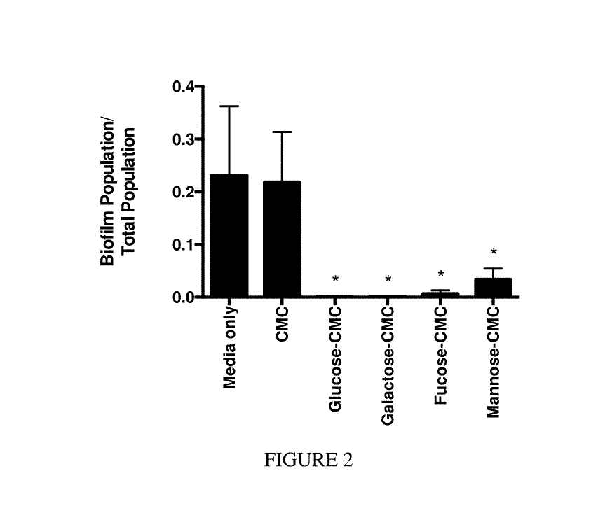

The polymers were evaluated in the biofilm inhibition assay compared to the nonfunctionalized CMC polymer. CMC alone did not have an inhibitory effect whereas each of the glycosylated polymers prevented or significantly inhibited biofilm formation (FIG. 2). These assay results demonstrate that the glycosylation of monosaccharides to CMC significantly changes the function of the polymer and inhibits biofilm formation of S. mutans.

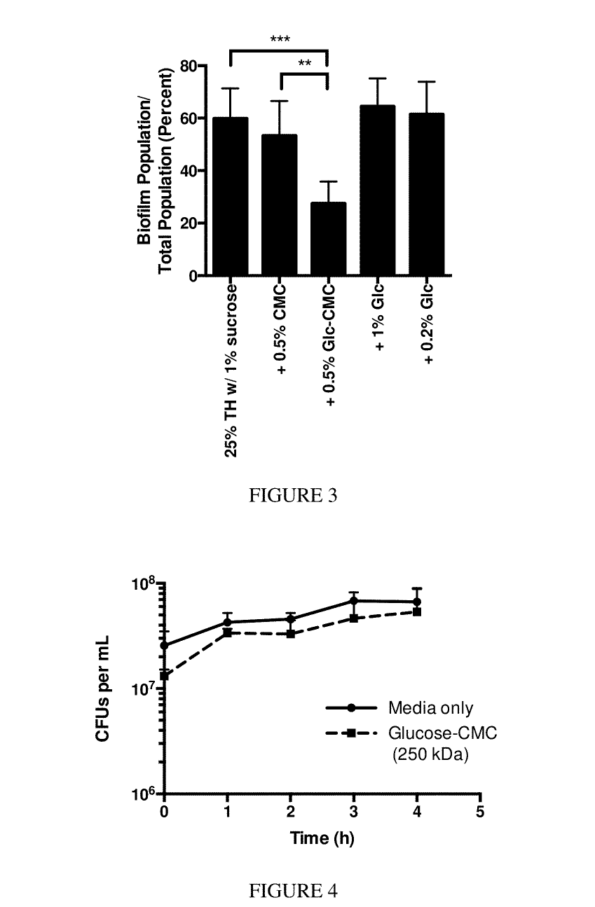

The biofilm inhibition assay was also employed to compare Glc-CMC and CMC mixed with unbound glucose (FIG. 3). Glc-CMC inhibited biofilm formation, but CMC mixed with 0.2% (w/v) unbound glucose did not inhibit biofilm formation, even when glucose was added at 1% (w/v) concentrations. These results suggest that glycans linked to mucins may exhibit different biological functions in their grafted form compared to their unbound form in solution. A possible explanation for the difference in function is the ability of microbes to metabolize monosaccharides in their soluble form. When covalently linked to CMC, glucose is unable to be metabolized by S. mutans. This is evidenced by the fact that the addition of Glc-CMC to the media did not enhance growth of S. mutans, which suggests that Glc-CMC is not a nutrient source (FIG. 4). Although Glc-CMC cannot be metabolized by S. mutans, glucose may still act as a biochemical signal to alter S. mutans physiology.

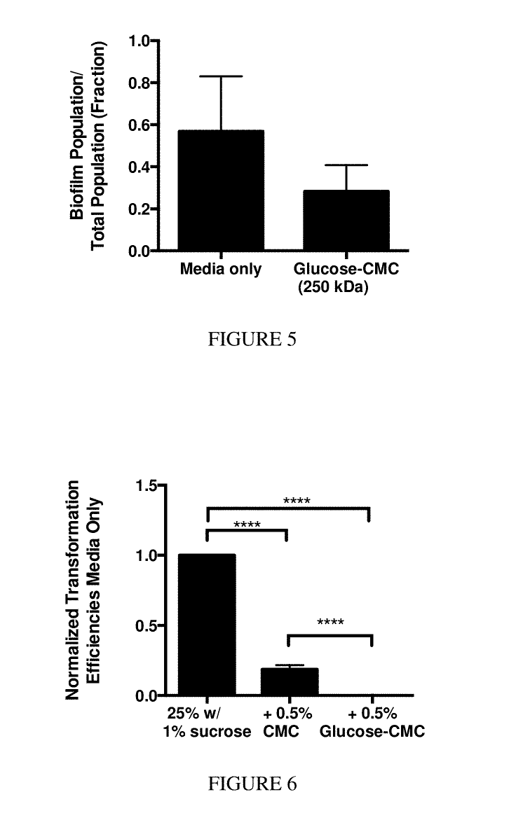

The effect of polymer length on function was also investigated. Two commercially available CMC polymers (90 kDa CMC and 250 kDa CMC) were employed to prepare Glc-CMC. Both Glc-CMC polymers were determined to have similar biofilm inhibitory effects in the S. mutans biofilm inhibition assay (FIGS. 3 and 5). From a functional perspective, these two polymers are not significantly different. However, the two different lengths of CMC are known to have varying degrees of viscosity at the same weight percent formulations. These results suggest that the functional component of glycan-grafted CMC can be independent from the mechanical properties. With these two independent design parameters, materials can be developed that have the same biological effects, but dramatically different viscoelastic profiles.

In addition, Glc-CMC was evaluated in the horizontal gene transfer assay, in which Glc-CMC prevented gene transfer between S. mutans and is much more effective than non-functionalized CMC alone (FIG. 6). These results suggest that the covalently bound glucose to CMC provides new biological function for the glycopolymer. This inhibitory activity is useful because it prevents the exchange of genetic material between microbes, which often leads to the acquisition of novel virulence and antibiotic resistance traits. Moreover, the ability of the exemplary polymers to inhibit additional virulence traits, such as natural transformation, is surprising and unexpected, as this property was previously identified to be specific to the salivary mucin MUC5B. These results suggest that the glycosylation of simple monosaccharides to a polymer backbone can impact multiple metabolic pathways of microbes and act as a broad-spectrum inhibitor of virulence traits without affecting cell populations.

The disclosed polymers represent a simple toolkit in which biochemical pathways can be investigated to understand which microbial cell surface receptors are activated or inhibited by mucin-like glycans. A possible master regulator is the phosphotransferase system (PTS), which is specific for the translocation of sugars across bacterial membranes. These two-component systems are useful for metabolizing nutrients, and are also implicated in the control and expression of virulence systems in bacteria. The PTS system revolves around using phosphoenolpyruvate (PEP) as an energy source. First discovered in E. coli, the PTS system has been identified in almost all bacterial species to date. In general, the PTS consists of two components: the intracellular Enzyme I (EI) and HPr, which are the main drivers in phosphorylation and downstream signaling, and Enzyme II (EII) as the cell surface transporter that is responsible for carbon source recognition. In E. coli, at least fifteen EII enzymes have been identified and are responsible for a wide range of carbohydrates. The EII cell surface transporters and sensors can be categorized into four major families depending on their specificities to saccharides. Phosporylation of EIIA subunits dictates what carbohydrates the bacteria preferentially react to and govern the overall genetic expression profile and physiology of the bacteria. The phosphorylation pathways of carbohydrate metabolism follow a tiered approach, with glucose generally being the preferred catabolite. As such, it is expected that the response of opportunistic pathogens is highly dependent on the type of saccharides exposed on the polymer backbone. In certain embodiments, the glucose-conjugated CMC polymers may induce responses from the broadest range of bacterial species due to glucose being a major catabolite-repressor.

As carbohydrate sensing dictates the metabolic response of bacteria, the presence and absence of sugars determines the virulence profile of pathogenic bacteria. In several bacterial species, expression of virulence genes is controlled by carbohydrate catabolite repression. For Clostridium difficile, which secretes colitis- and diarrhea-inducing exotoxins, the presence of monosaccharides inhibits expression of toxA and toxB genes. In the cavity causing bacteria, Streptococcus mutans, mutants without the EIIABC transporter exhibit higher levels of the virulence gene fructose hydrolase. These observations suggest that by providing bacteria with the correct carbohydrate signals, the expression of virulence genes may be modulated or inhibited.

### Equivalents and Scope

In the claims, articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process.

Furthermore, the invention encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group. It should it be understood that, in general, where the invention, or aspects of the invention, is/are referred to as comprising particular elements and/or features, certain embodiments of the invention or aspects of the invention consist, or consist essentially of, such elements and/or features. For purposes of simplicity, those embodiments have not been specifically set forth in haec verba herein.

It is also noted that the terms “comprising” and “containing” are intended to be open and permits the inclusion of additional elements or steps. Where ranges are given, endpoints are included. Furthermore, unless otherwise indicated or otherwise evident from the context and understanding of one of ordinary skill in the art, values that are expressed as ranges can assume any specific value or subrange within the stated ranges in different embodiments of the invention, to the tenth of the unit of the lower limit of the range, unless the context clearly dictates otherwise.

This application refers to various issued patents, published patent applications, journal articles, and other publications, all of which are incorporated herein by reference. If there is a conflict between any of the incorporated references and the instant specification, the specification shall control. In addition, any particular embodiment of the present invention that falls within the prior art may be explicitly excluded from any one or more of the claims. Because such embodiments are deemed to be known to one of ordinary skill in the art, they may be excluded even if the exclusion is not set forth explicitly herein. Any particular embodiment of the invention can be excluded from any claim, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, but rather is as set forth in the appended claims. Those of ordinary skill in the art will appreciate that various changes and modifications to this description may be made without departing from the spirit or scope of the present invention, as defined in the following claims.

1. A polymer of formula (I) 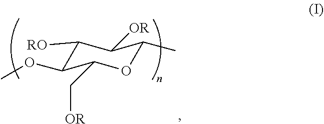
or a salt thereof, wherein:
each occurrence of R is independently hydrogen, —CH₂C(O)OH, —CH₂C(O)OR¹, —CH₂C(O)NHR¹, or R¹;
each occurrence of R¹ is independently a monosaccharide, oligosaccharide, or polysaccharide; and
n is equal to or greater than 20;
wherein the polymer has, on average, about 0.5 to about 2.0 R groups in each repeat unit that are —CH₂C(O)OH, —CH₂C(O)OR¹, or —CH₂C(O)NHR¹.

2. (canceled)

3. The polymer of claim 1, or a salt thereof, wherein R is hydrogen, —CH₂C(O)OH, —CH₂C(O)OR¹, or —CH₂C(O)NHR¹.

4. (canceled)

5. The polymer of claim 1 or a salt thereof, wherein the polymer has, on average, about 0.7 to about 1.2 R groups in each repeat unit that are —CH₂C(O)OH, —CH₂C(O)OR¹, or —CH₂C(O)NHR¹.

6. The polymer of any of claims 1, or a salt thereof, wherein R¹ is a monosaccharide or oligosacchadde,

7. (canceled)

8. The polymer of claim 1, or a salt thereof, wherein R¹ is lactose, a human milk oligosaccharide, or a mucin glycan.

9. (canceled)

10. The polymer of claim 1, or a salt thereof, wherein R¹ is glucose, galactose, mannose, fucose, sialic acid, glucosamine, galactosamine, mannosamine, fucosarnine, N-acetylglucosamine, or N-acetylgalactosamine.

11-12. (canceled)

13. The polymer of claim 1, or a salt thereof, wherein R is hydrogen, —CH₂C(O)OH,

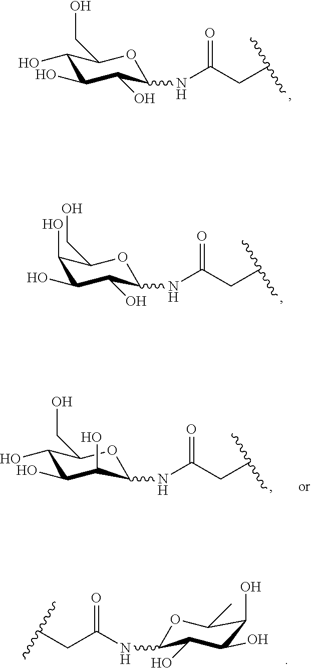

14. (canceled)

15. The polymer of claim 1, or a salt thereof, wherein the polymer has a molecular weight of up to 300,000 Da.

16. The polymer of claim 1, or a salt thereof, wherein the polymer has a molecular weight of up to 100,000 Da.

17-21. (canceled)

22. The polymer of claim 1, or a salt thereof, wherein the molecular weight of the R¹ groups comprise, by weight, about 10% of the molecular weight of the polymer.

23. A composition comprising the polymer of claim 1, or a salt thereof, and an acceptable carrier.

24. A pharmaceutical composition comprising the polymer of claim 1, or a salt thereof, and a pharmaceutically acceptable carrier.

25. A prebiotic composition comprising the polymer of claim 1, or a salt thereof, and a pharmaceutically acceptable carrier.

26. A food ct comprising the prebiotic composition of claim 25.

27. A lubricant comprising the polymer of claim 1, or a salt thereof.

28. A method of inhibiting microbial virulence, the method comprising contacting a microbe with the polymer of claim 1, or a salt thereof.

29. (canceled)

30. A method of inhibiting or preventing biofilm formation on a surface, the method comprising coating the surface with the polymer of claim 1, or a salt thereof.

31-33. (canceled)

34. A method of treating an infection in a subject in need thereof, the method comprising administering the polymer of claim 1, or a salt thereof.

35-37. (canceled)

38. A method of improving gastrointestinal health of a subject in need thereof, the method comprising administering the polymer of claim 1, or a salt thereof to the subject.

39-42. (canceled)

43. A method of preparing the polymer of claim 1, the method comprising coupling carboxymethyl cellulose with one or more monosaccharide, oligosaccharide, or polysaccharide to form the polymer.

44-49. (canceled)
